(19) United States
(12) Patent Application Publication
(10) Pub. No.: US 2016/0009610 A1
(43) Pub. Date: Jan. 14, 2016

(54) PROCESS FOR MAKING LINEAR LONG-CHAIN ALKANES FROM RENEWABLE FEEDSTOCKS USING CATALYSTS COMPRISING HETEROPOLYACIDS

(71) Applicant: E.I.DU PONT DE NEMOURS AND COMPANY, Wilmington, DE (US)

(72) Inventors: Annalisa Hargis, Wilmington, DE (US); Manish S. Kelkar, Wilmington, DE (US); Kenneth Chad Manning, Wilmington, DE (US); Joachim C. Ritter, Wilmington, DE (US); Sourav Kumar Sengupta, Wilmington, DE (US)

(21) Appl. No.: 14/771,676

(22) PCT Filed: Mar. 12, 2014
(86) PCT No.: PCT/US2014/023874

§ 371 Date: Aug. 31, 2015

Related U.S. Application Data
(60) Provisional application No. 61/782,172, filed on Mar. 14, 2013.
(60) Provisional application No. 61/782,198, filed on Mar. 14, 2013.

(51) Int. Cl. C07C 1/22; B01J 27/188; B01J 23/652

(52) U.S. Cl. CPC C07C 1/22; B01J 23/6527; B01J 27/188; C07C 2521/04; C07C 2523/652; C07C 2523/42; C07C 2523/30; C07C 2527/188; C07C 2527/185

(57) ABSTRACT

A hydrodeoxygenation process for producing a linear alkane from a feedstock comprising a saturated or unsaturated C₁₀₋₁₈ oxygenate that comprises an ester group, carboxylic acid group, carbonyl group and/or alcohol group is disclosed. This process comprises contacting the feedstock with (i) a catalyst comprising about 0.1% to about 10% by weight of a metal selected from Group IB, VIB, or VIII of the Periodic Table, and (ii) a heteropolyacid or heteropolyacid salt, at a temperature between about 150° C. to about 250° C. and a hydrogen gas pressure of at least about 300 psig. By contacting the feedstock with the catalyst and heteropolyacid or heteropolyacid salt under these temperature and pressure conditions, the C₁₀₋₁₈ oxygenate is hydrodeoxygenated to a linear alkane that has the same carbon chain length as the C₁₀₋₁₈ oxygenate.

This application claims the benefit of U.S. Provisional Application Nos. 61/782,172 and 61/782,198, each filed Mar. 14, 2013, both of which are incorporated herein by reference in their entireties.

### FIELD OF THE INVENTION

This invention is in the field of chemical processing. More specifically, this invention pertains to a process for producing linear long-chain alkanes from feedstocks comprising C₁₀₋₁₈ oxygenates such as fatty acids and triglycerides.

### BACKGROUND OF THE INVENTION

Long-chain alpha,omega-dicarboxylic acids (long-chain diacids, “LCDA”), i.e., those having a carbon chain length of 9 or higher, are used as raw materials in the synthesis of a variety of chemical products and polymers (e.g., long-chain polyamides). The types of chemical processes used to make long-chain diacids have a number of limitations and disadvantages, not the least of which is the fact that these processes are based on non-renewable petrochemical feedstocks. Also, the multi-reaction conversion processes used for preparing long-chain diacids generate unwanted by-products resulting in yield losses, heavy metal wastes and nitrogen oxides which need to be destroyed in a reduction furnace.

Given the high cost and increased environmental footprint left by fossil fuels and the limited petroleum reserves in the world, there is heightened interest in using renewable sources such as fats and oils obtained from plants, animals and microbes to make chemical products and polymers such as long-chain diacids.

Long-chain diacids can be made from long-chain alkanes, which in turn can be made by converting fatty acids and triglycerides via hydrodeoxygenation (HDO). The alkane products of this reaction not only can be used to produce long-chain diacids, but are also useful as fuel by itself or in a mixture with diesel from petroleum feedstocks.

Conventional deoxygenation processes for converting renewable feedstocks to long-chain alkanes include catalytic hydrodeoxygenation, catalytic or thermal decarboxylation, catalytic decarbonylation and catalytic hydrocracking. Commercially available deoxygenation reactions are typically operated under high pressure and temperature in the presence of hydrogen gas, rendering the process expensive. A few low pressure deoxygenation processes have also been described; however, such processes suffer from several disadvantages such as low activity, poor catalyst stability, and undesirable side reactions. Typically, these processes require a high temperature and result in a high degree of decarboxylation and decarbonylation, leading to shortening of chain length of the long-chain alkane products.

For example, U.S. Pat. Appl. Publ. No. 2012-0029250 discloses a deoxygenation process that produces pentadecane (C15:0) and heptadecane (C17:0) from palmitic acid (C16:0) and oleic acid (C18:1), respectively, via decarboxylation. This process also required a reaction temperature of at least 300° C. Besides resulting in products with carbon loss, the deoxygenation process also resulted in incompletely deoxygenated products such as stearic acid, unsaturated isomers of oleic acid, and branched products. The formation of decarboxylated as well as branched products was also observed using processes disclosed in U.S. Pat. Nos. 8,193,400 and 7,999,142.

U.S. Pat. No. 8,142,527 discloses a hydrodeoxygenation process to produce diesel fuel from vegetable and animal oils requiring a reaction temperature of at least 300° C. A hydrodeoxygenation process disclosed by U.S. Pat. No. 8,026,401 required a reaction temperature of at least 400° C.

Thus, there continues to be a need for hydrodeoxygenation processes that can be carried out under conditions of low temperature and pressure, and which reliably convert the fatty acids of oils and fats from renewable resources to long-chain, linear alkanes without substantial carbon loss.

### SUMMARY OF THE INVENTION

In one embodiment, the invention concerns a hydrodeoxygenation process for producing a linear alkane from a feedstock comprising a saturated or unsaturated C₁₀₋₁₈ oxygenate that comprises a moiety selected from the group consisting of an ester group, carboxylic acid group, carbonyl group and alcohol group. This process comprises contacting the feedstock with (i) a catalyst comprising about 0.1% to about 10% by weight of a metal selected from Group IB, VIB, or VIII of the Periodic Table, and (ii) a heteropolyacid or heteropolyacid salt, at a temperature between about 150° C. to about 250° C. and a hydrogen gas pressure of at least about 300 psig. By contacting the feedstock with the catalyst and heteropolyacid or heteropolyacid salt under these temperature and pressure conditions, the C₁₀₋₁₈ oxygenate is hydrodeoxygenated to a linear alkane that has the same carbon chain length as the C₁₀₋₁₈ oxygenate. Optionally, the hydrodeoxygenation process further comprises the step of recovering the linear alkane produced in the contacting step.

In a second embodiment, the C₁₀₋₁₈ oxygenate is a fatty acid or a triglyceride.

In a third embodiment, the feedstock comprises a plant oil or a fatty acid distillate thereof. The feedstock can comprise, for example, (i) a plant oil selected from the group consisting of soybean oil, palm oil and palm kernel oil; or (ii) a palm fatty acid distillate.

In a fourth embodiment, the catalyst comprises about 0.1% to about 6% by weight of platinum as the metal. The catalyst comprises about 0.25% to about 2% by weight of platinum as the metal in a fifth embodiment.

In a sixth embodiment, the heteropolyacid or heteropolyacid salt comprises tungsten. The heteropolyacid or heteropolyacid salt in this embodiment can further comprise phosphorus, for example.

In a seventh embodiment, the heteropolyacid salt is a heteropolyacid cesium salt.

In an eighth embodiment, the catalyst further comprises a solid support.

In a ninth embodiment, the feedstock is contacted with the catalyst and the heteropolyacid salt, and the catalyst is bound to the heteropolyacid salt.

In a tenth embodiment, the catalyst and the heteropolyacid or heteropolyacid salt are dry-mixed together before they are contacted with the feedstock. The catalyst and the heteropolyacid are dry-mixed together before they are contacted with the feedstock in an eleventh embodiment.

In a twelfth embodiment, the temperature is about 200° C. and the pressure is about 400 psig.

In a thirteenth embodiment, the molar yield of the hydrodeoxygenation process is less than 10% for a reaction product having a carbon chain length that is one or more carbon atoms shorter than the carbon chain length of the C₁₀₋₁₈ oxygenate.

### DETAILED DESCRIPTION OF THE INVENTION

The disclosures of all patent and non-patent literature cited herein are incorporated herein by reference in their entirety.

As used herein, the term “invention” or “disclosed invention” is not meant to be limiting, but applies generally to any of the inventions defined in the claims or described herein. The terms invention, disclosed invention and present invention are used interchangeably herein.

The terms “hydrodeoxygenation” (HDO), “hydrodeoxygenation process or reaction”, “deoxygenation process or reaction” and “hydrotreating” are used interchangeably herein. Hydrodeoxygenation as used herein refers to a chemical process in which hydrogen is used to reduce the oxygen content of an oxygen-containing organic compound such as an ester, carboxylic acid, ketone, aldehyde, or alcohol. Complete hydrodeoxygenation of such compounds typically yields an alkane, in which the carbon atom(s) that previously was bonded to an oxygen atom becomes hydrogen-saturated (i.e., the carbon atom has become “hydrodeoxygenated”). For example, hydrodeoxygenation of a carboxylic acid group or an aldehyde group yields a methyl group (—CH₃), whereas hydrodeoxygenation of a ketone group yields the internal carbon moiety —CH₂—.

The hydrodeoxygenation process as described herein also reduces alkene (C═C) and alkyne (C≡C) groups to C—C groups. Thus, the hydrodeoxygenation process can also be referred to as a process of reducing sites of unsaturation in organic compounds.

As used herein, hydrodeoxygenation does not refer to a process that reduces the oxygen content of a hydrocarbon through breaking a carbon-carbon bond, such as would occur with the removal of a carboxylic acid group (i.e., decarboxylation) or carbonyl group (i.e., decarbonylation). Neither does hydrodeoxygenation herein refer to a process that incompletely reduces an oxygenated carbon moiety (e.g., reduction of a carboxylic acid group to a carbonyl or alcohol group).

The terms “alkane”, “paraffin”, and “saturated hydrocarbon” are used interchangeably herein. An alkane as used herein refers to a chemical compound that consists only of hydrogen and carbon atoms, where the carbon atoms are bonded exclusively by single bonds (i.e., they are saturated compounds). Alkanes can be linear or branched and, therefore, have methyl groups at their termini.

The terms “linear alkane”, “straight-chain alkane”, “n-alkane”, and “n-paraffin” are used interchangeably herein and refer to an alkane that has only two terminal methyl groups and for which each internal (non-terminal) carbon atom is bonded to two hydrogens and two carbons. The short-hand formula for a linear alkane is CₙH₂ₙ₊₂. Linear alkanes differ from branched alkanes, which have three or more terminal methyl groups.

The term “C₁₀₋₁₈ oxygenate” as used herein refers to a linear chain of 10-18 carbon atoms in which one or more carbon atoms is bonded to an oxygen atom (i.e., one or more oxygenated carbons). Such oxygen-bonded carbon atoms are comprised in the C₁₀₋₁₈ oxygenate in the form of one or more alcohol, carbonyl, carboxylic acid, ester, and/or ether moieties. As would be understood in the art, the carboxylic acid, ester, and/or ether moieties, if present, would be located at one or both termini of the C₁₀₋₁₈ oxygenate.

Although the C₁₀₋₁₈ oxygenate can be 10, 11, 12, 13, 14, 15, 16, 17, or 18 carbon atoms in length, it typically has an even length of 10, 12, 14, 16, or 18 carbon atoms. Examples of C₁₀₋₁₈ oxygenates as referred to herein include, but are not limited to, esters, carboxylic acids, ketones, aldehydes and alcohols.

A “saturated C₁₀₋₁₈ oxygenate” as used herein refers to a C₁₀₋₁₈ oxygenate in which the constituent carbon atoms are linked to each other by single bonds (i.e., no double or triple bonds). An example of a saturated C₁₀₋₁₈ oxygenate is stearic acid (C18:0).

An “unsaturated C₁₀₋₁₈ oxygenate” as used herein refers to a C₁₀₋₁₈ oxygenate in which one or more double (alkene) or triple (alkyne) bonds are present in the carbon atom chain of the C₁₀₋₁₈ oxygenate. Examples of unsaturated C₁₀₋₁₈ oxygenates are oleic acid (C18:1) and linoleic acid (C18:2), which contain one and two double bonds, respectively.

An “ester group” as used herein refers to an organic moiety having a carbonyl group (C═O) (defined below) adjacent to an ether linkage. The general formula of an ester group is:

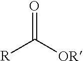

The R in the above ester formula refers to a linear chain of 9-17 carbon atoms; in this manner, the C═O carbon atom represents the tenth to eighteenth carbon atom of a C₁₀₋₁₈ oxygenate that contains an ester group. The R′ group refers to an alkyl or aryl group, for example. Examples of ester groups are found in mono- , di- and triglycerides which contain one, two, or three fatty acids, respectively, esterified to glycerol. With reference to the above formula, the R′ group of a monoglyceride would refer to the glycerol portion of the molecule. A linear alkane produced from an ester by the disclosed hydrodeoxygenation process contains the carbon atoms of the R group and the C═O group.

A “carboxylic acid group” or “organic acid group” as used herein refers to an organic moiety having a “carboxyl” or “carboxy” group (COOH). The general formula of a carboxylic acid group is:

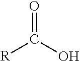

The R in the above carboxylic acid formula refers to a linear chain of 9-17 carbon atoms; in this manner, the carboxyl group (COOH) carbon atom represents the tenth to eighteenth carbon atom of a C₁₀₋₁₈ oxygenate that contains a carboxylic acid group. A linear alkane produced by the disclosed hydrodeoxygenation process retains the carboxyl group carbon atom (i.e., the product is not decarboxylated relative to the C₁₀₋₁₈ oxygenate substrate).

A “carbonyl group” as used herein refers to a carbon atom double-bonded to an oxygen atom (C═O). A carbonyl group can be located at either or both ends of the C₁₀₋₁₈ oxygenate; such a molecule could be referred to as an aldehyde. Alternatively, one or more carbonyl groups can be located within the carbon atom chain of the C₁₀₋₁₈ oxygenate; such a molecule could be referred to as a ketone.

An “alcohol group” as used herein refers to a carbon atom that is bonded to a hydroxyl (OH) group. One or more alcohol groups can be located at any carbon of the C₁₀₋₁₈ oxygenate (either or both ends, and/or one or more internal carbons of the C₁₀₋₁₈ oxygenate carbon chain).

The terms “feedstock” and “feed” are used interchangeably herein. A feedstock refers to a material comprising a saturated and/or unsaturated C₁₀₋₁₈ oxygenate. A feedstock may be a “renewable” or “biorenewable” feedstock, which refers to a material obtained from a biological or biologically derived source.

Examples of such feedstock are materials containing monoglycerides, diglycerides, triglycerides, free fatty acids, and/or combinations thereof, and include lipids such as fats and oils. These particular types of feedstocks, which can also be referred to as “oleaginous feedstocks”, include animal fats, animal oils, poultry fats, poultry oils, plant and vegetable fats, plant and vegetable oils, yeast oils, rendered fats, rendered oils, restaurant grease, brown grease, waste industrial frying oils, fish oils, fish fats, and combinations thereof. For feedstocks comprising fat or oil, it would be understood by one of skill in the art that all or most of the C₁₀₋₁₈ oxygenate is comprised in the feedstock in the form of an ester (fatty acid esterified to glycerol). Hydrodeoxygenation of such C₁₀₋₁₈ oxygenates according to the disclosed process involves the complete reduction of the ester group of the esterified fatty acid, which in part entails breaking the ester linkage between the fatty acid and the glycerol molecule.

Alternatively, a feedstock can refer to a petroleum- or fossil fuel-derived material comprising a saturated or unsaturated C₁₀₋₁₈ oxygenate.

The terms “fatty acid distillate” and “fatty acid distillate of an oil” as used herein refer to a composition comprising the fatty acids of a particular type of oil. For example, a palm fatty acid distillate comprises fatty acids that are present in palm oil. Fatty acid distillates commonly are byproducts of plant oil refining processes.

The terms “moiety”, “chemical moiety”, “functional moiety”, and “functional group” are used interchangeably herein. A moiety as used herein refers to a carbon group comprising a carbon atom bonded to an oxygen atom. Examples of a moiety as used herein include ester, carboxylic acid, carbonyl and alcohol groups.

The terms “percent by weight”, “weight percentage (wt %)” and “weight-weight percentage (% w/w)” are used interchangeably herein. Percent by weight refers to the percentage of a material on a mass basis as it is comprised in a composition or mixture. For example, percent by weight refers to the percentage of a metal by mass that is present in a catalyst as described herein. Except as otherwise noted, all the percentage amounts of metals or other materials disclosed herein refer to percent by weight of the metals or other materials in catalysts.

As used herein, “psig” (pound-force per square inch gauge) refers to a unit of pressure relative to atmospheric pressure at sea level. A psig of 30 represents an absolute pressure of 44.7 psi (i.e., 30 plus atmospheric psi of 14.7), for example.

The terms “catalyst” and “metal catalyst” are used interchangeably herein. The catalyst comprises a metal that increases the rate of C₁₀₋₁₈ oxygenate hydrodeoxygenation without itself being consumed or undergoing a chemical change. The catalyst is generally present in small amounts relative to the amounts of the reactants. In certain embodiments, the catalyst can also characterize a metal catalyst that is in the presence of a heteropolyacid or heteropolyacid salt. Such can be achieved by mixing the metal catalyst with the heteropolyacid or heteropolyacid salt (dry- or wet-mixing), or by binding the metal catalyst with the heteropolyacid or heteropolyacid salt.

The terms “Periodic Table” and “Periodic Table of the Elements” are used interchangeably herein.

The terms “solid support”, “support”, and “catalyst support” are used interchangeably herein. A solid support refers to the material to which an active metal is anchored or bound. Catalysts described herein that contain a solid support are examples of “supported metal catalysts” or “supported catalysts”. In certain embodiments, a metal catalyst may be supported on a heteropolyacid salt.

A “heteropolyacid” as used herein refers to a compound having a center element and peripheral elements to which oxygen is bonded (e.g., H₃PW₁₂O₄₀, where P is the central element and W is the peripheral element). Some heteropolyacid structures have been described; e.g., Keggin, Wells-Dawson and Anderson-Evans-Perloff structures. The center element in certain embodiments can be Si, P, Ge, As, B, Ti, Ce, Co, Ni, Al, Ga, Bi, Cr, Sn, or Zr. Examples of the peripheral element can be metals such as W, Mo, V, or Nb. A heteropolyacid is soluble in water. A “heteropolyacid salt” as used herein is a heteropolyacid that is ionically linked to a cation (e.g., cesium cation). A heteropolyacid salt can also be referred to as a “cation-exchanged heteropolyacid”. The heteropolyacid salts referred to herein are insoluble in water.

The terms “specific surface area”, “surface area”, and “solid support surface area” are used interchangeably herein. The specific surface area of a solid support is expressed herein as square meters per gram of solid support (m²/g). The specific surface area of the solid supports disclosed herein can be measured, for example, using the Brunauer, Emmett and Teller (BET) method (Brunauer et al., J. Am. Chem. Soc. 60:309-319; incorporated herein by reference).

The terms “impregnation” and “loading” are used interchangeably herein. Impregnation refers to the process of rendering a metal salt into a finely divided form or layer on a solid support. Generally, this process involves drying a mixture containing a solid support and a metal salt solution other water-dissolvable compound. The dried product can be referred to as a “pre-catalyst”. Where a metal catalyst is supported on a heteropolyacid salt, for example, the metal catalyst can be characterized as having been impregnated or loaded onto the heteropolyacid salt.

The terms “calcining” and “calcination” as used herein refer to a thermal treatment of a pre-catalyst. This process can convert a dried metal salt of a pre-catalyst to a metallic or oxide state, for example. The thermal treatment can be performed in either an inert or active atmosphere.

The terms “molar yield”, “reaction yield”, and “yield” are used interchangeably herein. Molar yield refers to the amount of a product obtained in a chemical reaction as measured on a molar basis. This amount can be expressed as a percentage; i.e., the percent amount of a particular product in all of the reaction products.

The terms “reaction mix”, “reaction mixture”, and “reaction composition” are used interchangeably herein. A reaction mix can minimally comprise a feedstock (substrate), metal catalyst, heteropolyacid or heteropolyacid salt, and a solvent. A reaction mix can describe the mix as it exists before or during application of the temperature and pressure hydrodeoxygenation conditions.

Disclosed herein is a hydrodeoxygenation process that can be carried out under conditions of low temperature and pressure, and which converts C₁₀₋₁₈ oxygenates in feedstocks to linear alkanes without substantial carbon loss. Therefore, the process produces fewer undesirable by-products and is more economical since it can be run under lower temperature and lower pressure conditions.

Embodiments of the disclosed invention concern a hydrodeoxygenation process for producing a linear alkane from a feedstock comprising a saturated or unsaturated C₁₀₋₁₈ oxygenate that comprises a moiety selected from the group consisting of an ester group, carboxylic acid group, carbonyl group and alcohol group. This process comprises contacting the feedstock with (i) a catalyst comprising about 0.1% to about 10% by weight of a metal selected from Group IB, VIB, or VIII of the Periodic Table, and (ii) a heteropolyacid or heteropolyacid salt, at a temperature between about 150° C. to about 250° C. and a hydrogen gas pressure of at least about 300 psig. By contacting the feedstock with the catalyst and heteropolyacid or heteropolyacid salt under these temperature and pressure conditions, the C₁₀₋₁₈ oxygenate is hydrodeoxygenated to a linear alkane that has the same carbon chain length as the C₁₀₋₁₈ oxygenate. Optionally, the hydrodeoxygenation process further comprises the step of recovering the linear alkane produced in the contacting step.

The feedstock used in certain embodiments of the disclosed invention may comprise a material comprising one or more monoglycerides, diglycerides, triglycerides, free fatty acids, and/or combinations thereof, and include lipids such as fats and oils. Examples of such feedstocks include fats and/or oil derived from animals, poultry, fish, plants, microbes, yeast, fungi, bacteria, algae, euglenoids and stramenopiles. Examples of plant oils include canola oil, corn oil, palm kernel oil, cheru seed oil, wild apricot seed oil, sesame oil, sorghum oil, soy oil, rapeseed oil, soybean oil, colza oil, tall oil, sunflower oil, hempseed oil, olive oil, linseed oil, coconut oil, castor oil, peanut oil, palm oil, mustard oil, cottonseed oil, camelina oil, jatropha oil and crambe oil. Other feedstocks include, for example, rendered fats and oil, restaurant grease, yellow and brown greases, waste industrial frying oil, tallow, lard, train oil, fats in milk, fish oil, algal oil, yeast oil, microbial oil, yeast biomass, microbial biomass, sewage sludge and soap stock.

Derivatives of oils such as fatty acid distillates are other examples of feedstocks that can be used in certain embodiments of the invention. Plant oil distillates (e.g., palm fatty acid distillate) are preferred examples of fatty acid distillates. A fatty acid distillate of any of the fats and oils disclosed herein may be used in certain embodiments of the invention.

The feedstock comprises a plant oil or a fatty acid distillate thereof in a preferred embodiment of the invention. In another preferred embodiment, the feedstock comprises (i) a plant oil selected from the group consisting of soybean oil, palm oil and palm kernel oil; or (ii) a palm fatty acid distillate (e.g., produced from refining crude palm oil).

Palm oil is derived from the mesocarp (pulp) of the fruit of the oil palm, whereas palm kernel oil is derived from the kernel of the oil palm. The fatty acids comprised in palm oil typically include palmitic acid (˜44%), oleic acid (˜37%), linoleic acid (˜9%), stearic acid and myristic acid. The fatty acids comprised in palm kernel oil typically include lauric acid (˜48%), myristic acid (˜16%), palmitic acid (˜8%), oleic acid (˜15%), capric acid, caprylic acid, stearic acid and linoleic acid. Soybean oil typically comprises linoleic acid (˜55%), palmitic acid (˜11%), oleic acid (˜23%), linolenic acid and stearic acid.

Fossil fuel-derived and other types of feedstocks that can be used in certain embodiments of the disclosed invention include petroleum-based products, spent motor oils and industrial lubricants, used paraffin waxes, coal-derived liquids, liquids derived from depolymerization of plastics such as polypropylene, high density polyethylene, and low density polyethylene; and other synthetic oils generated as byproducts from petrochemical and chemical processes.

Examples of other feedstocks are described in U.S. Pat. Appl. Publ. No. 2011-0300594, which is incorporated herein by reference.

The C₁₀₋₁₈ oxygenate comprised in the feedstock may be a fatty acid or a triglyceride. The feedstock may comprise one or more fatty acids that are in the free form (i.e., non-esterified) or that are esterified. Esterified fatty acids may be those comprised within a glyceride molecule (i.e., in a fat or oil) or fatty acid alkyl ester (e.g., fatty acid methyl ester or fatty acid ethyl ester), for example. The fatty acid(s) may be saturated or unsaturated. Examples of unsaturated fatty acids are monounsaturated fatty acids (MUFA) if only one double bond is present in the fatty acid carbon chain, and polyunsaturated fatty acids (PUFA) if the fatty acid carbon chain has two or more double bonds. The carbon chain length of a fatty acid C₁₀₋₁₈ oxygenate in the feedstock may be 10, 11, 12, 13, 14, 15, 16, 17, or 18 carbon atoms. Preferably, the carbon chain length is 10, 12, 14, 16, or 18 carbon atoms. Another preferred fatty acid length is 16-18 carbon atoms. Examples of fatty acids that can be in the feedstock are provided in Table 1.

TABLE 1
Examples of Saturated and Unsaturated Fatty Acids that May Be
Comprised in Feedstocks

| | | Shorthand |
|---|---|---|
| Common Name | Chemical Name | Notation |
| Capric | decanoic | 10:0 |
| Undecylic | undecanoic | 11:0 |
| Lauric | dodecanoic | 12:0 |
| Tridecylic | tridecanoic | 13:0 |
| Myristic | tetradecanoic | 14:0 |
| Myristoleic | tetradecenoic | 14:1 |
| pentadecylic | pentadecanoic | 15:0 |
| Palmitic | hexadecanoic | 16:0 |
| Palmitoleic | 9-hexadecenoic | 16:1 |
| | hexadecadienoic | 16:2 |
| Margaric | heptadecanoic | 17:0 |
| Stearic | octadecanoic | 18:0 |
| Oleic | cis-9-octadecenoic | 18:1 |
| Linoleic | cis-9,12-octadecadienoic | 18:2 omega-6 |
| gamma-linolenic | cis-6,9,12- | 18:3 omega-6 |
| | octadecatrienoic | |
| alpha-linolenic | cis-9,12,15- | 18:3 omega-3 |
| | octadecatrienoic | |
| Stearidonic | cis-6,9,12,15- | 18:4 omega-3 |
| | octadecatetraenoic | |

Although the oxygenates comprised in the feedstocks used in the disclosed invention have a length of 10-18 carbon atoms, other oxygenates with a carbon length outside this range may also be present in the feedstock. For example, the glycerides and free fatty acids of the fats and oils that can be used as feedstock may also contain carbon chains of about 8 to 24 carbon atoms in length. In other words, the feedstock need not comprise only C₁₀₋₁₈ oxygenates.

The C₁₀₋₁₈ oxygenates represented by lipids and free fatty acids comprise ester and carboxylic acid moieties, respectively. Other types of C₁₀₋₁₈ oxygenates may be comprised in the feedstock such as those C₁₀₋₁₈ oxygenates containing one or more carbonyl and/or alcohol moieties. Still other types of C₁₀₋₁₈ oxygenates may contain two or more of any of the above moieties. Examples include C₁₀₋₁₈ oxygenates comprising two or more alcohol moieties (e.g., diols), carbonyl moieties (e.g., diketones or dialdehydes), carboxylic acid moieties (dicarboxylic acids), or ester moieties (diesters). C₁₀₋₁₈ oxygenates comprising alcohol and carbonyl moieties (e.g., hydroxyketones and hydroxyaldehydes), alcohol and carboxylic acid moieties (e.g., hydroxycarboxylic acids), alcohol and ester moieties (e.g., hydroxyesters), carbonyl and carboxylic acid moieties (e.g., keto acids), or carbonyl and ester moieties (e.g., keto esters) are other example components of feedstocks that can be used in embodiments of the disclosed invention.

The feedstock may contain one or more C₁₀₋₁₈ oxygenates linked together by two or more ester and/or ether linkages. Such C₁₀₋₁₈ oxygenates are unlinked from each other during the disclosed hydrodeoxygenation process; the removal of oxygen from such molecules destroys the ester and/or ether linkages. Similarly, the fatty acid C₁₀₋₁₈ oxygenates as contained in a glyceride feedstock are unlinked from the glycerol component of the glyceride during the disclosed hydrodeoxygenation process since the fatty acid ester linkages are destroyed by the removal of oxygen. Therefore, different types of linear alkanes can be produced from feedstocks containing two or more different C₁₀₋₁₈ oxygenates, even if the C₁₀₋₁₈ oxygenates are linked by ester and/or ether linkages. All these types of C₁₀₋₁₈ oxygenates may be constituent components of the feedstock.

The linear chain of the C₁₀₋₁₈ oxygenate is not linked to any alkyl or aryl branches via a carbon-carbon bond from one of the carbon atoms of the linear chain. For example, while palmitic acid is a C₁₀₋₁₈ oxygenate in certain embodiments, palmitic acid having an alkyl group substitution (e.g., 15-methyl palmitic acid) at one of its —CH₂— moieties is not a type of C₁₀₋₁₈ oxygenate as described herein. The hydrodeoxygenation process of the invention does not involve isomerization events that involve removing and/or adding carbon-carbon bonds to a carbon of the C₁₀₋₁₈ oxygenate. Therefore, branched alkane products such as isodecanes, isododecanes, isotetradecanes, isohexadecanes and isooctadecanes are not produced.

The C₁₀₋₁₈ oxygenate may constitute the feedstock itself in certain embodiments of the disclosed invention. An example of such a feedstock is a pure or substantially pure preparation of a particular fatty acid. Alternatively, the feedstock may comprise multiple separate C₁₀₋₁₈ oxygenates (i.e., distinct molecules that are not linked to each other). Mixtures of any of the above feedstocks may be used as co-feed components in the disclosed hydrodeoxygenation process.

A linear alkane is produced from a saturated or unsaturated C₁₀₋₁₈ oxygenate in the disclosed hydrodeoxygenation process. The linear alkanes produced in certain embodiments include decane, undecane, dodecane, tridecane, tetradecane, pentadecane, hexadecane, heptadecane and octadecane, where those of these linear alkanes having an even carbon atom number are produced in preferred embodiments.

As discussed above, hexadecane is a linear alkane produced in certain embodiments of the disclosed hydrodeoxygenation process. Various C₁₆ oxygenates can be used as feedstock to produce hexadecane, including hexadecanol (e.g., cetyl alcohol), hexadecyl aldehyde, hexadecyl ketone, palmitic acid, palmityl palmitate, and/or any other C₁₆ oxygenate in which one or more carbon atoms of the C16 chain is bonded to an oxygen atom, for example. The feedstock in certain embodiments may comprise any of these various C₁₆ oxygenates. For example, the feedstock may be an oil or fat comprising palmitic acid (i.e., contain a palmitoyl group) or palmitoleic acid (i.e., contain a 9-hexadecenoyl group).

Octadecane is a linear alkane produced in certain embodiments of the disclosed hydrodeoxygenation process. Various C₁₈ oxygenates can be used as feedstock to produce octadecane, including octadecanol (e.g., stearyl alcohol), octadecyl aldehyde, octadecyl ketone, stearic acid, stearyl stearate, and/or any other C₁₈ oxygenate in which one or more carbon atoms of the C18 chain is bonded to an oxygen atom, for example. The feedstock in certain embodiments may comprise any of these various C₁₈ oxygenates. For example, the feedstock may be an oil or fat comprising stearic acid (i.e., contain a stearoyl group), oleic acid (i.e., contain a 9-octadecenoyl group), or linoleic acid (i.e., contain a 9, 12-octadecadienoyl group).

The molar yield of the linear alkane is at least about 25% in certain embodiments of the disclosed invention. In other embodiments, the molar yield of the linear alkane is at least about 10%, 11%, 12%, 13%, 14%, 15%, 16%, 17%, 19%, 20%, 21%, 22%, 23%, 24%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%. For example, where lauric acid is the C₁₀₋₁₈ oxygenate comprised in the feedstock for the process, the molar yield of dodecane is at least about 25%.

The carbon chain length of the linear alkane product of the disclosed hydrodeoxygenation process is the same carbon chain length of the C₁₀₋₁₈ oxygenate. For example, where the C₁₀₋₁₈ oxygenate is palmitic acid, the resulting linear alkane is hexadecane; both palmitic acid and hexadecane have a carbon chain length of sixteen carbon atoms. The linear alkane produced in the disclosed process therefore represents the completely hydrogen-saturated, reduced form of the C₁₀₋₁₈ oxygenate in the feedstock. For example, the disclosed hydrodeoxygenation process produces decane from capric acid; dodecane from lauric acid; tetradecane from myristic acid and myristoleic acid; hexadecane from palmitic acid and palmitoleic acid; and octadecane from stearic acid, oleic acid and linoleic acid. These linear alkanes are produced whether the fatty acids are free or esterified. A C₁₀₋₁₈ oxygenate that is linked to one or more other components via ester and/or ether linkages yields a linear alkane during the disclosed process that represents the completely hydrogen-saturated, reduced form of the C₁₀₋₁₈ oxygenate.

In certain embodiments of the disclosed invention, the molar yield is less than about 10% for a reaction product having a carbon chain length that is one or more carbon atoms shorter than the carbon chain length of the C₁₀₋₁₈ oxygenate. For example, where lauric acid is the C₁₀₋₁₈ oxygenate comprised in the feedstock for the process, the molar yield of undecane which has a chain length of eleven carbon atoms is less than about 10%. In other embodiments, the molar yield is less than about 1%, 2%, 3%, 4%, 5%, 6%, 7%, 8%, or 9% for a reaction product having a carbon chain length that is one or more carbon atoms shorter than the carbon chain length of the C₁₀₋₁₈ oxygenate. The low level of such byproducts using the disclosed invention reflects a low level of carbon loss from the C₁₀₋₁₈ oxygenate by decarboxylation and/or decarbonylation events during the hydrodeoxygenation reaction. Therefore, the disclosed process does not significantly break carbon-carbon bonds of the C₁₀₋₁₈ oxygenate.

The molar yield of other types of byproducts in certain embodiments of the disclosed invention is less than about 1%, 2%, 3%, 4%, 5%, 6%, 7%, 8%, 9%, 10%, 11%, 12%, 13%, 14%, or 15%. Such other byproducts include products that represent incompletely reduced forms of the C₁₀₋₁₈ oxygenate that retain one or more oxygenated carbon atoms (e.g., alcohol group, carbonyl group, carboxylic acid group, ester group), and/or one or more points of unsaturation. Examples of byproducts include dodecanol and lauryl laurate in certain embodiments of the hydrodeoxygenation process using lauric acid in the feedstock.

The hydrodeoxygenation process of the invention can be tested with respect to its ability to convert dodecanol into dodecane. In other words, a hydrodeoxygenation process for converting C₁₆ or C₁₈ oxygenates to alkanes can be tested using lauric acid or dodecanol as the feedstock; such processes when tested on lauric acid or dodecanol can have molar yields of dodecane as listed above for linear alkanes. Similarly, such processes when tested on lauric acid or dodecanol can have molar yields of byproducts less than about 1%, 2%, 3%, 4%, 5%, 6%, 7%, 8%, 9%, 10%, 11%, 12%, 13%, 14%, or 15%.

The linear alkanes produced in the disclosed process can be isolated by close-cut distillation, for example. If necessary, selective adsorption with molecular sieves can be used to further purify the linear alkanes from those reaction byproducts that are bulkier than the linear alkanes. Molecular sieves can comprise synthetic zeolites having a series of central cavities interconnected by pores. The pores have diameters large enough to permit passage of linear alkanes, but not large enough to allow passage of branched byproducts. Commercial isolation processes using molecular sieves include IsoSiv™ (Dow Chemical Company), Molex™ (UOP LLC) and Ensorb™ (Exxon Mobil Corporation), for example.

The disclosed invention includes the step of contacting the feedstock comprising a C₁₀₋₁₈ oxygenate with a catalyst and a heteropolyacid or heteropolyacid salt at a temperature between about 150° C. to about 250° C. and a hydrogen gas pressure of at least about 300 psig.

The step of contacting the feedstock with the catalyst and a heteropolyacid or heteropolyacid salt may be performed in a reaction vessel or any other enclosure known in the art that allows performing a reaction under controlled temperature and pressure conditions. For example, the contacting step is performed in a packed bed reactor, such as a plug flow, tubular or other fixed bed reactor. It should be understood that the packed bed reactor may be a single packed bed or comprise multiple beds in series and/or in parallel. Alternatively, the contacting step can be performed in a slurry reactor, including batch reactors, continuously stirred tank reactors, and/or bubble column reactors. In slurry reactors, the catalyst may be removed from the reaction mixture by filtration or centrifugal action. The size/volume of the reaction vessel should be suitable for handling the chosen amount of feedstock and catalyst.

The contacting step may be performed in a continuous or batch processing system as known in the art. A continuous process may be multi-stage using a series of two or more reactors in series. Fresh hydrogen may be added at the inlet of each reactor in this type of system. A recycle stream may also be used to help maintain the desired temperature in each reactor. The reactor temperature may also be controlled by controlling the fresh feedstock temperature and the recycle rate.

In certain embodiments, the contacting step may comprise agitating or mixing the feedstock and catalyst and heteropolyacid or heteropolyacid salt before and/or while the reaction components are subjected to the above temperature and hydrogen gas pressure conditions. Agitation can be performed using a mechanical stirrer, or in a slurry reactor system, for example.

The order in which the feedstock may be contacted with the catalyst and heteropolyacid or heteropolyacid salt can vary. For example, the feedstock may be contacted with a preparation (dry-mix or wet mix) containing both the catalyst and heteropolyacid or heteropolyacid salt. Alternatively, for example, the feedstock may first be contacted with the catalyst in a reaction mix, after which the heteropolyacid or heteropolyacid salt is added to the reaction mix. Alternatively still, for example, the feedstock may first be contacted with the heteropolyacid or heteropolyacid salt in a reaction mix, after which the catalyst is added to the reaction mix.

The contacting step in certain embodiments may be performed in a solvent, such as an organic solvent or water. The solvent may consist of one type of solvent that is pure or substantially pure (e.g., >99% or >99.9% pure) or comprise two or more different solvents mixed together. The solvent may be homogeneous (e.g., single-phase) or heterogeneous (e.g., two or more phases). In a preferred embodiment, the feedstock and the catalyst and heteropolyacid or heteropolyacid salt are contacted in an organic solvent. The organic solvent used in certain embodiments may be non-polar or polar. The organic solvent comprises tetradecane, hexadecane, or dodecane in another embodiment. Alternatively, the organic solvent may be another alkane such as one having a chain length of 6 to 18 carbon atoms. The organic solvent may be selected on the basis of its ability to dissolve hydrogen. For example, the solvent can have a relatively high solubility for hydrogen so that substantially all the hydrogen provided by the hydrogen gas pressure is in solution before and/or during the disclosed hydrodeoxygenation process. The heteropolyacid or heteropolyacid salt is not soluble in an organic solvent in certain embodiments.

Certain embodiments of the invention comprise using a solvent comprising tetradecane and hexadecane. Examples of such a solvent have a tetradecane-to-hexadecane ratio of about 15:1, 16:1, 17:1, 18:1, 19:1, or 20:1, where the ratio is determined on a weight basis (e.g., grams). A ratio of about 17:1 tetradecane-to-hexadecane is preferred in certain embodiments. Solvents having these relative amounts of tetradecane and hexadecane may be capable of enhancing product yields in certain of the hydrodeoxygenation reactions disclosed herein.

The contacting step of the process of the invention is performed at a temperature between about 150° C. to about 250° C. and a hydrogen gas pressure of at least about 300 psig. The temperature in certain embodiments may be about 150° C., 160° C., 170° C., 180° C., 190° C., 200° C., 210° C., 220° C., 230° C., 240° C., or 250° C. Alternatively, the temperature is between about 150° C. to about 200° C. The temperature is about 200° C. and the pressure is about 400 psig in other embodiments. The hydrogen gas pressure in certain embodiments may be between about 300 psig to about 1000 psig, about 300 psig to about 500 psig, between about 350 psig to about 450 psig, or at about 400 psig. Alternatively, the hydrogen gas pressure in certain embodiments is between about 300 psig to about 1000 psig.

In certain embodiments of the disclosed invention, the feedstock and catalyst and heteropolyacid or heteropolyacid salt are contacted in the above temperature and hydrogen pressure conditions for about 2, 3, 4, 5, 6, 7, 8, 9, 10, 11, 12, 13, 14, 15, 16, 17, 18, 19 or 20 hours. The feedstock and catalyst can be subjected to the temperature and hydrogen pressure conditions for a continuous period of time, for example.

In certain embodiments, the feedstock is contacted with hydrogen to form a feedstock/hydrogen mixture in advance of contacting the feedstock with the catalyst and heteropolyacid or heteropolyacid salt. In other embodiments, a solvent or diluent is added to the feedstock in advance of contacting the feedstock with hydrogen and/or catalyst and heteropolyacid or heteropolyacid salt. For example, after forming a feedstock/solvent mixture, it may then be contacted with hydrogen to form a feedstock/solvent/hydrogen mixture which is then contacted with the catalyst and heteropolyacid or heteropolyacid salt.

A wide range of suitable catalyst concentrations may be used in the disclosed process, where the amount of catalyst per reactor is generally dependent on the reactor type. For a fixed bed reactor, the volume of catalyst per reactor will be high, while in a slurry reactor, the volume will be lower. Typically, in a slurry reactor, the catalyst will make up 0.1 to about 30 wt % of the reactor contents.

The disclosed invention includes contacting the feedstock comprising a C₁₀₋₁₈ oxygenate with a catalyst comprising (i) about 0.1% to about 10% by weight of a metal selected from Group IB, VIB, or VIII of the Periodic Table. Such a metal may be copper, silver, or gold, which are Group IB metals; chromium, molybdenum, or tungsten, which are Group VIB metals; or iron, ruthenium, osmium, cobalt, rhodium, iridium, nickel, palladium, or platinum, which are Group VIII metals. In certain preferred embodiments, the metal is platinum.

In additional embodiments, a second metal can be included in the catalyst such as about 0.5% to about 15% by weight of tungsten, rhenium, molybdenum, vanadium, manganese, zinc, chromium, germanium, tin, titanium, gold or zirconium.

The catalyst in certain embodiments may comprise about 0.1%, 0.25%, 0.5%, 0.75%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5% or 10% by weight of one or more of any of the metal selected from Group IB, VIB, or VIII of the Periodic Table. The second metal, if included in the catalyst, can be at about 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0.%, 14.5%, or 15.0% by weight. In certain embodiments, the catalyst comprises no more than one, two, or three different metals. In certain other embodiments, the catalyst comprises platinum as the only metal; however, metals that are present in a heteropolyacid or heteropolyacid salt (e.g., tungsten) can be used with such catalysts.

The catalyst in certain embodiments of the invention comprises platinum as the metal. The catalyst comprises about 0.1% to about 6% by weight of platinum as the metal in certain preferred embodiments. The catalyst in certain other preferred embodiments comprises about 0.25% to about 2% by weight of platinum as the metal. In other embodiments, the catalyst comprises about 0.1% to about 2%, about 0.25% to about 2%, about 0.1% to about 1%, or about 0.25% to about 1% by weight of platinum as the metal.

The catalyst further comprises a solid support in certain embodiments of the invention. Various solid supports as known in the art can be comprised in the catalyst, including one or more of WO₃, Al₂O₃ (alumina), TiO₂ (titania), Ti0₂—Al₂O₃, ZrO₂, tungstated ZrO₂, SiO₂, SiO₂—Al₂O₃, SiO₂—TiO₂, V₂O₅, MoO₃, or carbon (e.g., activated carbon), for example. In certain preferred embodiments, the solid support comprises Al₂O₃ or carbon. The solid support may therefore comprise an inorganic oxide, metal oxide or carbon. Other examples of solid supports that may be used include clay (e.g., montmorillonite) and zeolite (e.g., H—Y zeolite). The support material used in the catalyst such as those described above may be basic (≧pH 9.5), neutral, weakly acidic (pH between 4.5 and 7.0), or acidic (≦pH 4.5). Additional examples of solid supports that can be used in certain embodiments of the disclosed invention are described in U.S. Pat. No. 7,749,373 which is herein incorporated by reference.

The solid support used in certain embodiments of the disclosed invention may be porous, thereby increasing the surface area onto which the metal catalyst is attached. In certain preferred embodiments, the solid support comprises pores and has (i) a specific surface area that is at least 10 m²/g and optionally less than or equal to 280 m²/g, wherein the pores have a diameter greater than 500 angstroms and the pore volume of the support is at least 10 ml/100 g; or (ii) a specific surface area that is at least 50 m²/g and optionally less than or equal to 280 m²/g, wherein the pores have a diameter greater than 70 angstroms and the pore volume of the support is at least 30 ml/100 g.

Thus, the specific surface area of the solid support in certain embodiments is about or at least about 50, 100, 110, 120, 130, 140, 150, 160, 170, 180, 190, 200, 210, 220, 230, 240, 250, 260, 270, 280, 290, 300, 400, 500, 600, 700, 800, 900, 1000, 1100, 1200, 1300, 1400 or 1500 m²/g. Preparing a porous solid support with a particular specific surface area can be performed by modulating pore diameter and volume as known in the art (e.g., Trimm and Stanislaus, Applied Catalysis 21:215-238; Kim et al., Mater. Res. Bull. 39:2103-2112; Grant and Jaroniec, J. Mater. Chem. 22:86-92).

The solid support in certain embodiments may have a mean particle size and/or a particle size distribution D50 of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 microns. “D50”, which is a median value, is the diameter (typically in microns) that splits the distribution of support particles with half above and half below this diameter.

Solid supports for preparing the catalysts used in certain embodiments of the disclosed invention are available from a number of commercial sources, including Johnson Matthey, Inc. (West Deptford, N.J.), BASF (Iselin, N.J.), Evonik (Calvert City, Ky.) and Sigma-Aldrich (St. Louis, Mo.), for example. Regarding support materials from Johnson Matthey (JM), alumina particles designated #32 (JM #32) herein have a mean particle size of 8 microns and a surface area of 300 m²/g, alumina particles designated #33 (JM #33) herein have a mean particle size of 18 microns and a surface area of 180 m²/g, carbon particles designated #23 (JM #23) herein have a mean particle size of 30 microns and a surface area of 1500 m²/g, carbon particles designated #25 (JM #25) herein have a mean particle size of 35 microns and a surface area of 900 m²/g, and carbon particles designated #26 (JM #26) herein have a mean particle size of 30 microns and a surface area of 1500 m²/g. Regarding materials from Evonik, designations F 101 XR, F 101 KYF, F 1181 XR and F 1181 KYF herein refer to carbon loaded with 1% or 2% by weight platinum. Further regarding materials from Evonik, the carbon of F 101 XR herein is a powder having a particle size distribution D50 of 28 microns and a surface area of 1090 m²/g, the carbon of F 101 KYF herein is a powder having a particle size distribution D50 of 28 microns and a surface area of 1090 m²/g, the carbon of F 1181 XR herein is a powder having a particle size distribution D50 of 18 microns and a surface area of 1140 m²/g, the carbon of F 1181 KYF herein is a powder having a particle size distribution D50 of 18 microns and a surface area of 1140 m²/g, the carbon of “EVONIK 5% Pt+5% Bi on Carbon” herein is a powder having a particle size distribution D50 of 28 microns and a surface area of 1120 m²/g, and the carbon of “EVONIK 1% Pt+0.1% Cu on Carbon” herein is a powder having a particle size distribution D50 of 28 microns and a surface area of 1120 m²/g.

Supported metal catalysts in certain embodiments can be in the form of particles such as shaped particles. Catalyst particles can be shaped as cylinders, pellets, spheres, or any other shape. Cylinder-shaped catalysts may have hollow interiors, with or without one or more reinforcing ribs. Other particle shapes that may be used include trilobe, cloverleaf, cross, “C”-shaped, rectangular- and triangular-shaped tubes, for example. Alternatively, supported metal catalyst may be in the form of powder or larger sized cylinders or tablets.

The metal catalyst can be used with a supported heteropolyacid or heteropolyacid salt in certain embodiments. The heteropolyacid or heteropolyacid salt may be supported on SiO₂, Al₂O₃, or any other support material such as those disclosed herein. In certain embodiments, platinum can be loaded onto, and thus be supported by, a supported heteropolyacid or heteropolyacid salt. For example, platinum could be loaded onto (i) WPA supported on SiO₂, Al₂O₃, or carbon, or (ii) Cs—WPA supported on SiO₂, Al₂O₃, or carbon.

Other examples of metal catalyst compositions that can be used in certain embodiments of the disclosed invention are described in U.S. Pat. Appl. Publ. Nos. 2011-0300594 and 2012-0029250, and U.S. Pat. No. 8,084,655, all of which are incorporated herein by reference.

The catalyst can be prepared using any of a variety of ways known in the art (e.g., Pinna, 1998, Catalysis Today 41:129-137; Catalyst Preparation: Science and Engineering, Ed. John Regalbuto, Boca Raton, Fla.: CRC Press, 2006; Mul and Moulijn, Chapter 1: Preparation of supported metal catalysts, In: Supported Metals in Catalysis, 2nd Edition, Eds. J. A. Anderson and M. F. Garcia, London, UK: Imperial College Press, 2011; Acres et al., The design and preparation of supported catalysts, In: Catalysis: A Specialist Periodical Report, Eds. D. A. Dowden and C. C. Kembell, London, UK: The Royal Society of Chemistry, 1981, vol. 4, pp. 1-30). It is desirable that the catalyst prepared by a chosen method be active, selective, recyclable, and mechanically and thermochemically stable during the disclosed hydrodeoxygenation process.

The impregnation of the metal onto the solid support in preparing certain catalysts for use in the disclosed process may be performed by mixing the solid support with a metal salt solution, drying this mixture at a suitable temperature (e.g., 100 to 120° C.) for a suitable amount of time to obtain a dried product, and then calcining the dried product at a suitable temperature (e.g., 300 to 400° C.) for a suitable amount of time. The supported metal catalyst prepared by this procedure may then be impregnated with another metal, if desired. Another way of loading more than one metal, if desired, to a support may be performed by mixing the solid support with a metal salt solution, drying this mixture as above, mixing the dried product with another metal salt solution, drying this mixture as above, and then calcining the dried product as above. Either of the above procedures can be adapted accordingly to load additional metals onto the solid support, if desired.

The present invention includes the step of contacting the feedstock comprising a C₁₀₋₁₈ oxygenate with a heteropolyacid or heteropolyacid salt. The heteropolyacid as known in the art is a compound having a center element and peripheral elements to which oxygen is bonded. The center element can be Si, P, Ge, As, B, Ti, Ce, Co, Ni, Al, Ga, Bi, Cr, Sn, or Zr, for example. Examples of the peripheral element can be metals such as W, Mo, V, or Nb. Some heteropolyacid structures have been described; e.g., Keggin, Wells-Dawson and Anderson-Evans-Perloff structures.

The heteropolyacid in certain embodiments can have a chemical structure represented by one of the following formulae:

HₖXM₁₂O₄₀ or (I)

AⱼHₖXM₁₂O₄₀, wherein (II)
X (center element) is Si, P, Ge, As, B, Ti, Ce, Co, Ni, Al, Ga, Bi, Cr, Sn, or Zr;
M (peripheral element) is independently Mo, W, V, or Nb;
A (see formula II) is an alkali metal element, alkaline earth metal element, organic amine cation, or a combination thereof;
k is 0-4;
j is 0-4; and
at least one of j and k is greater than 0.

It would be understood in the art that formula II represents a heteropolyacid salt or a cation-exchanged heteropolyacid (where A in formula II is the cation). Examples of A (the cation) in formula II include metal ions such as lithium, sodium, potassium, cesium, magnesium, barium, copper, rubidium, thallium, gold or gallium ions; thus the heteropolyacid salt can be a heteropolyacid metal salt. Other examples of A in formula II include onium groups such as ammonium (NH₄⁺) and organic amine. The cation in certain embodiments of formula II may be selected on the basis that the cation-exchanged heteropolyacid is acidic and insoluble in water. Thus, the catalyst composition in certain embodiments comprises a heteropolyacid salt that is acidic and insoluble in water. Such a heteropolyacid salt can be a cesium-exchanged heteropolyacid (heteropolyacid cesium salt), for example. It would be understood in the art that the cesium ion in such heteropolyacid salts represents the A group in formula II. The A group in certain other embodiments can alternatively be potassium or ammonium. In certain embodiments, j+k≦4 in formula II.

The heteropolyacid or heteropolyacid salt in certain embodiments of the disclosed invention comprises tungsten (W). It would be understood in the art that the tungsten in such heteropolyacids represents peripheral element M in either formula I or II. The heteropolyacid or heteropolyacid salt in certain embodiments comprises phosphorus (P). It would be understood in the art that the phosphorus in such heteropolyacids represents center element X in either formula I or II.

Examples of heteropolyacids that can be used include H₃PW₁₂O₄₀.(phosphotungstic acid, abbreviated herein as “WPA”) and H₄SiW₁₂O₄₀, which both follow formula I. Examples of heteropolyacid salts include Cs_{2.5}H_{0.5}PW₁₂O₄₀ (phosphotungstic acid cesium salt, abbreviated herein as “Cs—WPA”) and Cs_{2.5}H_{0.5}SiW₁₂O₄₀, which both follow formula II.

The heteropolyacid in certain embodiments can be either anhydrous or hydrated. Hydrated heteropolyacids (i.e., containing crystallization water) include H₃PW₁₂O₄₀.(H₂O)ₓ and H₄SiW₁₂O₄₀.(H₂O)ₓ, for example, where x is equal to or greater than 1. However, in certain embodiments, a hydrated heteropolyacid is dehydrated before using it to prepare the disclosed catalyst compositions.

Various amounts of the heteropolyacid or heteropolyacid salt can be used when contacting the feedstock with the catalyst and the heteropolyacid or heteropolyacid salt. For example, the percent by weight of the heteropolyacid or heteropolyacid salt in a reaction mix may be at least about 1.0%, 1.1%, 1.2%, 1.3%, 1.4%, 1.5%, 1.6%, 1.7%, 1.8%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% (percent of the total weight of the reaction mix). The amount of heteropolyacid or heteropolyacid salt in a reaction mix can be stated differently, for example, such as with respect to the amount of supported metal catalyst present in the reaction mix.

In certain embodiments, the amount of heteropolyacid or heteropolyacid salt in a reaction mix can be set as a percentage of the weight of the supported metal catalyst in the reaction mix. For example, if 0.10 g of supported metal catalyst is used, a “loading” of 50% of a heteropolyacid or heteropolyacid salt would mean that 0.05 g of heteropolyacid or heteropolyacid salt is used in the reaction. A loading of about 10%, 20%, 30%, 40%, 50%, or 60% of a heteropolyacid or heteropolyacid salt can be used in certain embodiments following this measurement scheme.

In certain embodiments, the amount of a heteropolyacid or heteropolyacid salt in a reaction mix can be set with respect to the amount of tungsten (W) that can be introduced to a reaction using a particular heteropolyacid or salt thereof. For example, if phosphotungstic acid (WPA) is used as a heteropolyacid in the reaction, the desired amount of W to be loaded with the supported metal catalyst would be equal to a certain percentage of the weight of the supported metal catalyst. As an example, if 0.1 g of supported metal catalyst is used in a reaction mix, and the desired “loading” of W is 2% using WPA, then about 0.026 g of WPA would be used to provide 0.002 g of W, which is 2% of the weight of 0.1 g. The amount of WPA to use in this example (0.026 g) is based on there being about 76.6 wt % W in one molecule of WPA. A loading of about 1%, 2%, 3%, 4%, 5%, 6%, 7%, 8%, 9%, 10%, 20%, 30%, 40%, 50%, 60%, 70%, 80%, 90%, or 100% of a W into a reaction mix using a heteropolyacid (e.g., WPA) or heteropolyacid salt can be used in certain embodiments following this measurement scheme.

In certain embodiments, the feedstock is contacted with the catalyst and the heteropolyacid salt, and the catalyst is bound to the heteropolyacid salt. This is an example in which a heteropolyacid salt effectively serves as a support material. In this role in certain embodiments, the heteropolyacid salt can alter or enhance the activity of a metal catalyst, and/or may itself contribute to the catalytic activity of the metal catalyst.

Preparation of the heteropolyacid salt as a metal catalyst support may follow any procedure known in the art, or any of the procedures disclosed herein. The heteropolyacid salt in certain embodiments may be calcined (or not calcined) before it is used as a support. Loading of the heteropolyacid in certain embodiments may be done using wet-impregnation, for example, with a metal catalyst salt solution (e.g., ammonium platinum nitrate). For example, after application and removal of a catalyst metal salt solution to a heteropolyacid salt, the wet-impregnated heteropolyacid salt may be dried at a suitable temperature (e.g., about 100° C.) and then calcined at a suitable temperature (e.g., about 350° C.) for a suitable amount of time (e.g., about 3 hours).

In certain embodiments in which the metal catalyst is bound to a heteropolyacid salt, the metal catalyst may be loaded at about 0.25%, 0.5%, 1.0%, or 2.0% by weight of the final catalyst composition. The metal catalyst can be platinum, for example, in any of these embodiments. In other examples, the heteropolyacid salt may be a phosphotungstic acid salt such as phosphotungstic acid cesium salt. Any catalytic metal-bound heteropolyacid salt as disclosed herein may be used in a hydrodeoxygenation reaction where it is at least about 1 to 10%, 1 to 5%, 2 to 5%, 3 to 5% or 4 to 5% (e.g., about 4.7%) by weight of the total weight of the reaction mix, for example.

The catalyst and heteropolyacid or heteropolyacid salt are dry-mixed together before they are contacted with the feedstock in certain embodiments of the disclosed hydrodeoxygenation process. In other embodiments, the catalyst and heteropolyacid are dry-mixed together before they are contacted with the feedstock.

In certain embodiments, the catalyst and heteropolyacid or heteropolyacid salt can be prepared by mixing the catalyst with the heteropolyacid or heteropolyacid salt under dry conditions. In other words, the mixing is performed without the introduction of water or any solution. For example, a dry amount of a metal catalyst and a dry amount of a heteropolyacid or heteropolyacid salt are placed together and ground down to a powder. The resulting powder contains fine particles of the metal catalyst and the heteropolyacid or heteropolyacid salt. This mixing can also be referred to as intimate mixing. Thus, in certain embodiments, the catalyst composition comprises a dry mixture of the metal catalyst and the heteropolyacid or heteropolyacid salt. This mixture can also be referred to as a fine mixture, intimate mixture, or a powder mixture.

In alternative embodiments, the mixing can be performed by mixing together catalyst and heteropolyacid or heteropolyacid salt that have each already been rendered into a powder (i.e., metal catalyst powder is mixed with heteropolyacid powder). Still alternatively, one component not previously rendered as a powder may be placed with the other component that has already been rendered as a powder, after which the first component is ground down (e.g., metal catalyst powder and non-powderized heteropolyacid salt may be placed together and subject to grinding/powderization).

Depending on the size of the operation, dry-mixing can be performed using an industrial grinder as known in the art, or on a smaller scale using a mortar and pestle, for example. Dry-mixing can be performed in certain embodiments such that a certain amount of pressure or force is applied by the mixing device to the particles being mixed. For example, the mixing device may apply force of about 100 to 500 pounds (e.g., about 300 pounds) to the components being mixed. The force applied to the components being mixed does not need to be uniformly applied throughout the mixing process.

The mixing process can be carried out for any suitable amount of time. For example, mixing of the metal catalyst and heteropolyacid components may be performed for at least about 1, 2, 3, 4, 5, 6, 7, 8, 9, or 10 minutes. Catalyst compositions prepared by dry-mixing may be used in a hydrodeoxygenation reaction immediately after preparation (e.g. within about 15 or 30 minutes), or may be stored for later use, preferably in an inert atmosphere.

The catalyst composition prepared by dry-mixing the metal catalyst and heteropolyacid or heteropolyacid salt components may comprise particles having an average mesh size (U.S. standard scale) of about 140, 130, 120, 110, 100, 90, 80, 70, 60, 50, or 40, for example (or about 140 to 40 mesh). In certain embodiments, the average particle size (e.g., longest length dimension) is about 150, 160, 170, 180, 190, 200, 210, 220, 230, 240 or 250 microns (or about 150-250 microns). Thus, a dry mixture, fine mixture, or intimate mixture can refer to a mix of metal catalyst and heteropolyacid/heteropolyacid salt particles having an average mesh size or particle size as listed above.

The term “dry mixture” is meant to refer to the state of the catalyst composition upon its production. In this sense, the catalyst composition in certain embodiments can be one that was produced by dry-mixing. It would be understood that use of the dry mixture in the disclosed hydrodeoxygenation process exposes it to liquids such as solvents, certain feedstocks and/or certain products.

The heteropolyacid salt used in the various embodiments disclosed herein can be prepared by any means known in the art. Alternatively, it can be prepared by the following steps.

An aqueous heteropolyacid solution can be prepared by first dehydrating the heteropolyacid (if hydrated) using heat (e.g., about 60° C.) and/or vacuum for a suitable amount of time (e.g., about 2 hours), for example. The dehydrated heteropolyacid is then dissolved in water to provide an aqueous solution. The heteropolyacid salt is precipitated from this solution to render a heteropolyacid salt.

The salt used to precipitate the heteropolyacid (“heteropolyacid-precipitating salt”) can first be dried under high heat (e.g., about 420° C.) and/or a vacuum for a suitable amount of time (e.g., about 2 hours), for example, before preparing a solution of the salt.

The heteropolyacid-precipitating salt in certain embodiments can contain the element or group represented by A in formula II. For example, the salt can be one or more of a carbonate, hydroxide, sulfate, or sulfide of any of the elements or groups represented by A in formula II. Cs₂CO₃ is an example of a carbonate that could be used as the heteropolyacid-precipitating salt.

The precipitated heteropolyacid salt in certain embodiments may be dried using heat (e.g. about 100-150° C. or 120° C.) and/or vacuum conditions to remove water for a suitable amount of time (e.g., about 2 hours). This dried product may further be calcined in certain embodiments at a temperature of about 250-350° C. (e.g., about 300° C.) for a suitable amount of time (e.g., about 1 hour).

The linear alkanes produced by the disclosed invention are suitable for use in producing long-chain diacids by fermentation. For example, the linear alkanes may be fermented, individually or in combination, to linear dicarboxylic acids of 10 (decanedioic acid), 12 (dodecanedioic acid), 14 (tetradecanedioic acid), 16 (hexadecanedioic acid), or 18 (octadecanedioic acid) carbons in length. Methods and microorganisms for fermenting linear alkanes to linear dicarboxylic acids are described, for example, in U.S. Pat. Nos. 5,254,466; 5,620,878; 5,648,247, and U.S. Pat. Appl. Publ. Nos. 2011-0300594, 2005-0181491 and 2004-0146999 (all of which are herein incorporated by reference). Methods for recovering linear dicarboxylic acids from fermentation broth are also known, as disclosed in some of the above references and also in U.S. Pat. No. 6,288,275 and International Pat. Appl. Publ. No. W02000-020620.

### EXAMPLES

The invention is further defined in the following Examples. It should be understood that these Examples, while indicating preferred aspects of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Catalyst Synthesis: 2% Tungsten/5% Platinum on Al₂O₃

This Example describes the impregnation of tungsten (W) on an alumina (Al₂O₃)-supported platinum (Pt) catalyst. The resulting alumina-supported tungsten/platinum catalyst was used in the low temperature/low pressure hydrodeoxygenation processes described in Examples 2 and 3.

Two wt % tungsten was loaded on a 5 wt % alumina-supported platinum catalyst (Pt/Al₂O₃) using a wet impregnation method. To achieve tungsten impregnation on the support, 0.083 g of ammonium tungstate pentahydrate (Strem Chemicals, Newburyport, Mass.; lot no. 19424200) was dissolved in 2 mL of deionized water. This solution was then added to 2.92 g of dry alumina-supported 5% platinum catalyst (Pt/Al₂O₃) powder from Johnson Matthey, Inc. (West Deptford, N.J.; #33 in the Heterogeneous Catalyst Screening Kit, Catalyst Designation B301099-5). This Pt/Al₂O₃ powder has a moisture content of 1%, uniform metal location, a surface area of 180 m²/g, a mean particle size of 15 microns, and a nitrobenzene activity of 200 mL H₂/15 min. The mixture-solution was vortexed for about 5 minutes. The sample was then dried overnight for about 16 hours in a vacuum oven at 110° C. under a vacuum of 20 mm Hg. A small nitrogen bleed was used to assist in the removal of water vapor during this drying process. The sample was then cooled to room temperature before calcining it for 3 hours at 350° C.

Thus, a 2% tungsten/5% platinum alumina-supported catalyst was obtained.

### Example 2

### Selective Hydrodeoxygenation of Lauric Acid to Dodecane in a 600-cc Reactor

This Example describes the selective hydrodeoxygenation of lauric acid to dodecane using the alumina-supported tungsten/platinum catalyst prepared in Example 1 (2% W on 5% Pt/alumina). This process was carried out under conditions including a temperature of 200° C. and a pressure of 500 psig (pound-force per square inch gauge).

30.5 g of lauric acid (Sigma Aldrich, St. Louis, Mo.; >99%, lot. no. MKBG4553V), 253.0 g of tetradecane (Alfa Aesar, Ward Hill, Mass.; 99+%, lot. no. E09Y007), 15.3 g of hexadecane (Sigma Aldrich, 99.9%, lot. no. 26396JMV) and 3.19 g of the alumina-supported tungsten/platinum catalyst prepared in Example 1 were added to a 600-cc Hastelloy Parr® pressure reactor equipped with a mechanical stirrer. The mechanical stirrer was set to a rotation speed of 700 rpm. The reactor was purged with nitrogen gas six times by pressurizing the reactor to about 400 psig each time and then depressurizing it. The reactor was then purged with hydrogen gas six times by pressurizing the reactor to 200 psig and then depressurizing it.

After these purging cycles, the reactor was pressurized to about 100 psig of hydrogen and heated to 200° C. Once the reactor was equilibrated at 200° C., the reactor pressure was raised to the experimental set point of 500 psig. A sample of the above input material (lauric acid with tetradecane, hexadecane and the W/Pt catalyst) was collected through a sample port immediately after the reaction conditions (200° C. and 500 psig) were reached. Additional samples were collected every hour for the next five hours. After 6 hours at 200° C., the reactor was allowed to cool down to 50° C. and was held under those conditions overnight.

The reactor was reheated again to 200° C. the following day and the hydrogen pressure was adjusted to 500 psig. Again, a sample was collected immediately after the reaction conditions (200° C. and 500 psig) were reached and every hour thereafter for the next six hours. After 6 hours, the reactor was allowed to cool down to room temperature and was depressurized to ambient pressure before dismantling it and collecting the reaction mixture.

All the collected samples were diluted with tetrahydrofuran and filtered through a standard 0.45-micron disposable filter. The filtered samples were then analyzed by a GC/FID (gas chromatography/flame ionization detector) to identify the components thereof and to measure the concentrations of the reactants and products. The individual components were identified by matching the retention times of the components with those of certain calibration standards. The hexadecane that was included in the reaction was used as an internal standard to determine the concentrations of each of the individual components.

With respect to the final product of the reaction, the conversion of lauric acid was observed to be about 95%, while the molar yield of dodecane was about 65%. The molar yield was about 12% for dodecanol, about 11% for lauryl laurate, and about 2% for undecane.

These results demonstrate that a feedstock comprising the C₁₂ oxygenate, lauric acid, can be used to produce a linear alkane via a hydrodeoxygenation process that employs a W/Pt/alumina catalyst under conditions of low temperature and pressure. The hydrodeoxygenation process mostly produced a completely deoxygenated product (dodecane) with a small amount of certain by-products.

Specifically, the low production of undecane (C₁₁) demonstrates that only a very low level of carbon loss through lauric acid decarboxylation occurred during the process. The high level of dodecane produced with a relatively low amount of the side products dodecanol and lauryl laurate demonstrates that the process efficaciously deoxygenated the carboxylic acid moiety of the lauric acid feedstock.

Given the results described in Example 8-11, inclusion of a heteropolyacid or heteropolyacid salt would be applicable to the reaction procedure described in this Example.

### Example 3

### Selective Hydrodeoxygenation of Lauric Acid to Dodecane in a 20-cc Multi-Reactor System

This Example describes the selective hydrodeoxygenation of lauric acid to dodecane using the alumina-supported tungsten/platinum catalyst prepared in Example 1 (2% W on 5% Pt/alumina). This process was carried out under conditions including a temperature of 200° C. and a pressure of 400 psig.

The hydrodeoxygenation reaction was performed in an Endeavor® reactor system containing eight stainless steel reaction vessels. Each vessel has a volume of about 25 mL and is equipped with mechanical stirring. A 20-mL glass vial is used as a liner for each reactor in this system.

0.20 g of lauric acid (Sigma Aldrich, >99%, lot. no. MKBG4553V), 1.71 g of tetradecane (Alfa Aesar, 99+%, lot. no. E09Y007), 0.10 g of hexadecane (Sigma Aldrich, 99.9%, lot. no. 26396JMV) and 0.10 g of the alumina-supported tungsten/platinum catalyst prepared in Example 1 were added to a 20-mL glass vial used in one of the reaction vessels in the Endeavor® reaction system. The system was sealed and connected to a high pressure gas manifold. The reactor was purged with nitrogen gas four times by pressurizing the reactor to 400 psig each time and then depressurizing it. The reactor was then purged with hydrogen gas three times by pressurizing the reactor to 400 psig and then depressurizing it.

After the purging cycles, the reactor was pressurized to 100 psig of hydrogen and heated to 200° C. Once the reactor temperature reached 200° C., more hydrogen was added to the reactor to raise its pressure to the experimental set point of 400 psig. The reaction was carried out isothermally for four hours before switching off the heat and cooling the reactor down to below 50° C. For the entire length of the reaction, the pressure was maintained at the 400 psig set point by adding hydrogen whenever the pressure dropped below 399 psig.

After the reactor was cooled down, the glass vial used for the reaction was removed from the reactor and centrifuged at 2000 rpm for 5 minutes. The resulting sample was decanted off to separate the catalyst (solid sample) from the rest of the reaction mixture (liquid sample). The liquid sample was further diluted with tetrahydrofuran and filtered through a standard 0.45-micron disposable filter. The filtered liquid sample was then analyzed by a GC/FID to identify the components thereof and to measure the concentrations of the reactants and products. The individual components were identified by matching the retention times of the components with those of certain calibration standards. The hexadecane that was included in the reaction was used as an internal standard to determine the concentrations of each of the individual components.

With respect to the final product of the reaction, the conversion of lauric acid was observed to be about 99%, while the molar yield of dodecane was about 79% (Table 2, seventh row). The molar yield was about 1% for dodecanol, about 2% for lauryl laurate, and about 3% for undecane.

These results further demonstrate that a hydrodeoxygenation process employing a W/Pt/alumina catalyst under conditions of low temperature and pressure can be used to produce a linear alkane from the C₁₂ oxygenate, lauric acid. The hydrodeoxygenation process mostly yielded the completely deoxygenated, full-length product, dodecane, with a very small amount of by-products. The low level of undecane produced indicates that there was little carbon loss during the process, while the low levels of dodecanol and lauryl laurate indicate that the carboxylic acid moiety of the lauric acid was efficiently deoxygenated.

Given the results described in Example 8-11, inclusion of a heteropolyacid or heteropolyacid salt would be applicable to the reaction procedure described in this Example.

### Example 4

### Selective Hydrodeoxygenation of Lauric Acid to Dodecane in a 20-cc Multi-Reactor System Using Various Catalysts

This Example describes the selective hydrodeoxygenation of lauric acid to dodecane using various other catalysts aside from the 2% W on 5% Pt/alumina described above. For those other catalysts containing platinum and tungsten, they differed from each other in the amount of platinum and tungsten contained therein and/or in the manner in which the platinum and tungsten were applied to the alumina support during catalyst preparation. Catalysts containing other metals such as nickel, copper, bismuth, molybdenum, palladium, manganese, chromium and vanadium were also tested.

Other catalysts were prepared and tested for their ability to catalyze deoxygenation of lauric acid to dodecane following the 20-cc reaction procedure described in Example 3. These other catalysts are listed in Table 2 and were prepared in a manner similar with the protocol described in Example 1 for preparing 2% W on 5% Pt/alumina. In general, catalysts containing tungsten and platinum were prepared by impregnating tungsten onto alumina-supported platinum (“Pt/alumina”) or by impregnating tungsten and platinum onto alumina. Alumina was impregnated with other metals (vanadium, palladium, manganese, chromium, molybdenum) in the preparation of other catalysts. Both the Pt/alumina (refer to Example 1) and alumina were from Johnson Matthey, Inc.

TABLE 2
Molar Selectivity and Molar Yield of Dodecane and Undecane from the
Conversion of Lauric Acid Using Various Catalysts^{a}

| | Lauric | | | | |
|---|---|---|---|---|---|
| | Acid | | | | |
| | Con- | Dodecane | | Undecane | |
| Catalyst | version^{b} | Selectivity^{c} | Yield | Selectivity^{c} | Yield |
| 0.4% W on 1% | 45% | 1% | 0% | 1% | 0% |
| Pt/alumina | | | | | |
| 0.8% W on 1% | 27% | 3% | 1% | 2% | 1% |
| Pt/alumina | | | | | |
| 2% W on 1% | 28% | 4% | 1% | 2% | 1% |
| Pt/alumina | | | | | |
| 0.4% W on 2% | 27% | 7% | 2% | 1% | 0% |
| Pt/alumina | | | | | |
| 0.8% W on 2% | 30% | 11% | 3% | 2% | 1% |
| Pt/alumina | | | | | |
| 2% W on 2% | 42% | 24% | 10% | 3% | 1% |
| Pt/alumina | | | | | |
| 2% W on 5% | 99% | 80% | 79% | 3% | 3% |
| Pt/alumina | | | | | |
| 2% Pt on 2% W | 56% | 5% | 3% | 6% | 3% |
| on alumina | | | | | |
| 2% Pt on 5% W | 50% | 13% | 6% | 9% | 5% |
| on alumina | | | | | |
| 2% W on 2% Pt | 36% | 5% | 2% | 6% | 2% |
| on alumina | | | | | |
| 5% W on 2% Pt | 56% | 20% | 11% | 9% | 5% |
| on alumina | | | | | |
| 2% W and 2% Pt | 39% | 8% | 3% | 3% | 1% |
| on alumina | | | | | |
| 5% W and 2% Pt | 44% | 9% | 4% | 3% | 1% |
| on alumina | | | | | |
| 5% W on 1% | 77% | 18% | 13% | 2% | 1% |
| Pt/alumina | | | | | |
| 7.5% W on 1% | 75% | 15% | 11% | 2% | 1% |
| Pt/alumina | | | | | |
| 10% W on 1% | 59% | 12% | 7% | 2% | 1% |
| Pt/alumina | | | | | |
| 5% W on 2% | 93% | 61% | 57% | 3% | 3% |
| Pt/alumina | | | | | |
| 5% W on 2% | 100% | 69% | 69% | 3% | 3% |
| Pt/alumina | | | | | |
| 5% W on 2% | 99% | 56% | 56% | 2% | 2% |
| Pt/alumina | | | | | |
| 7.5% W on 2% | 98% | 39% | 38% | 2% | 2% |
| Pt/alumina | | | | | |
| 10% W on 2% | 93% | 30% | 28% | 2% | 2% |
| Pt/alumina | | | | | |
| 5% W on Ni | 47% | 4% | 2% | 2% | 1% |
| 10% W on Ni | 36% | 6% | 2% | 3% | 1% |
| 1% Pt on Ni | 57% | 3% | 2% | 5% | 3% |
| 0.5% Pt on Ni | 67% | 3% | 2% | 10% | 7% |
| 5% W on 1% Pt | 34% | 7% | 2% | 4% | 2% |
| on Ni | | | | | |
| 5% W on 0.5% | 44% | 5% | 2% | 6% | 2% |
| Pt on Ni | | | | | |
| 1% Pt and 0.1% | 18% | 6% | 1% | 6% | 1% |
| Cu on carbon | | | | | |
| 5% Pt and 5% Bi | 13% | 5% | 1% | 9% | 1% |
| on carbon | | | | | |
| 2% Mo on 2% | 85% | 2% | 2% | 2% | 2% |
| Pd on alumina | | | | | |
| 2% W on 2% Pd | 32% | 3% | 1% | 4% | 1% |
| on alumina | | | | | |
| 2% Cr on 2% Pd | 32% | 2% | 1% | 4% | 1% |
| on alumina | | | | | |
| 2% Mn on 2% | 33% | 2% | 1% | 3% | 1% |
| Pd on alumina | | | | | |
| 2% V on 2% Pd | 27% | 3% | 1% | 4% | 1% |
| on alumina | | | | | |

^{a}Reactions performed with listed catalyst under the conditions described in Example 3.
^{b}Percent moles of lauric acid that converted to products.
^{c}Percent moles of lauric acid that converted into dodecane or undecane.

The catalysts listed in Table 2 as “0.4% W on 1% Pt/alumina”, “0.8% W on 1% Pt/alumina”, “2% W on 1% Pt/alumina”, “0.4% W on 2% Pt/alumina”, “0.8% W on 2% Pt/alumina”, “2% W on 2% Pt/alumina”, “5% W on 1% Pt/alumina”, “7.5% W on 1% Pt/alumina”, “10% W on 1% Pt/alumina”, “5% W on 2% Pt/alumina”, “7.5% W on 2% Pt/alumina” and “10% W on 2% Pt/alumina” were prepared following the procedures described in Example 1. However, different concentrations of ammonium tungstate pentahydrate were used accordingly to load tungsten onto Pt/alumina containing either 1% or 2% by weight platinum.

The catalysts listed in Table 2 as “2% Pt on 2% W on alumina”, “2% Pt on 5% W on alumina”, “2% W on 2% Pt on alumina”, and “5% W on 2% Pt on alumina” were prepared by sequentially impregnating platinum and tungsten onto alumina. The catalysts 2% Pt on 2% W on alumina and 2% Pt on 5% W on alumina were prepared by first loading tungsten onto alumina (forming alumina-supported tungsten) followed by loading platinum onto the alumina-supported tungsten. Oppositely, catalysts 2% W on 2% Pt on alumina and 5% W on 2% Pt on alumina were prepared by first loading platinum onto alumina (forming alumina-supported platinum) followed by loading tungsten onto the alumina-supported platinum. Each metal was loaded onto support in a manner similar to the process described in Example 1. Briefly, a salt of tungsten (ammonium tungstate pentahydrate) or platinum (tetraamine platinum nitrate) was dissolved in deionized water and then mixed with alumina. The mixture was dried for about 16 hours at 110° C. under a vacuum of 20 mm Hg, cooled, and then calcined for 3 hours at 350° C., yielding either alumina-supported tungsten or platinum. Impregnation of the next metal (either tungsten or platinum) was then performed via this same procedure, but using the alumina-supported tungsten or platinum produced from the first step as the loading target. The production of these particular catalysts thus involved two calcining steps. Using appropriate metal salts, similar processes were used accordingly to prepare the catalysts listed in Table 2 as “2% Mo on 2% Pd on alumina”, “2% W on 2% Pd on alumina”, “2% Cr on 2% Pd on alumina”, “2% Mn on 2% Pd on alumina” and “2% V on 2% Pd on alumina”.

The catalysts listed in Table 2 as “2% W and 2% Pt on alumina” and “5% W and 2% Pt on alumina” were prepared using one calcining step, as follows. A tetraamine platinum nitrate solution was mixed with alumina and then dried for about 16 hours at 110° C. under a vacuum of 20 mm Hg. The dried product was then mixed with an ammonium tungstate pentahydrate solution and then dried as above. The dried product, which contained both platinum and tungsten with alumina, was then calcined for 3 hours at 350° C. Using appropriate metal salts and carbon as a support, similar processes were used accordingly to prepare the catalysts listed in Table 2 as “1% Pt and 0.1% Cu on carbon” and “5% Pt and 5% Bi on carbon”.

The results listed in Table 2 indicate that several catalysts in addition to 2% W on 5% Pt/alumina are capable of catalyzing hydrodeoxygenation of lauric acid to dodecane at 200° C. and 400 psig. For example, the products of reactions using catalysts 2% W on 2% Pt/alumina (Table 2, sixth row) and 5% W on 2% Pt on alumina (Table 2, eleventh row) had molar yields of dodecane that were more than two times higher than the respective molar yields of undecane. Also, reactions using several of the catalysts in Table 2 had molar yields of dodecane greater than 25%. For example, reactions using three separate preparations of a 5% W on 2% Pt/alumina catalyst had dodecane yields of at least 56%, with undecane yields of 3% or lower.

Given the results described in Example 8-11, inclusion of a heteropolyacid or heteropolyacid salt would be applicable to the reaction procedure described in this Example.

### Example 5

### Selective Hydrodeoxygenation of Lauric Acid to Dodecane in a 20-cc Multi-Reactor System Using Catalysts Containing Alumina of Various pH Levels

This Example describes the selective hydrodeoxygenation of lauric acid to dodecane using catalysts containing 5% W and 2% Pt supported on an acidic, weakly acidic, neutral, or basic alumina solid support.

The catalysts in this example were prepared using a particular type of alumina (acid, weakly acidic, neutral, or basic) following the methodology described in Example 4 for catalysts designated as “5% W on 2% Pt on alumina”. These catalysts were then used in a lauric acid hydrodeoxygenation reaction under the conditions described in Example 3. The results of these reactions are listed in Table 3.

TABLE 3
Molar Selectivity and Molar Yield of Dodecane and Undecane from the
Conversion of Lauric Acid Using Catalysts Containing Alumina of
Various pH^{a}

| | Lauric | | | | |
|---|---|---|---|---|---|
| | Acid | | | | |
| | Con- | Dodecane | | Undecane | |
| Catalyst | version^{b} | Selectivity^{c} | Yield | Selectivity^{c} | Yield |
| 5% W on 2% Pt on | 53% | 41% | 22% | 4% | 2% |
| basic alumina | | | | | |
| 5% W on 2% Pt on | 63% | 45% | 28% | 4% | 2% |
| neutral alumina | | | | | |
| 5% W on 2% Pt on | 66% | 48% | 32% | 4% | 3% |
| weakly acidic | | | | | |
| alumina | | | | | |
| 5% W on 2% Pt on | 64% | 48% | 30% | 3% | 2% |
| acidic alumina | | | | | |

^{a}Reactions performed with listed catalyst under the conditions described in Example 3.
^{b}Percent moles of lauric acid that converted to products.
^{c}Percent moles of lauric acid that converted into dodecane or undecane.

The results listed in Table 3 indicate that catalysts containing 5% W and 2% Pt supported on acidic, weakly acidic, neutral, or basic alumina were all capable of catalyzing hydrodeoxygenation of lauric acid to dodecane at 200° C. and 400 psig. All the reactions yielded 22%-32% dodecane, while yielding 3% or less of undecane. These results suggest that catalysts containing various amounts of tungsten and platinum supported on alumina of various pH levels are capable of catalyzing C₁₀₋₁₈ oxygenate hydrodeoxygenation.

Given the results described in Example 8-11, inclusion of a heteropolyacid or heteropolyacid salt would be applicable to the reaction procedure described in this Example.

### Example 6

### Selective Hydrodeoxygenation of Lauric Acid to Dodecane in a 20-cc Multi-Reactor System Using a Six-Hour Reaction Period

This Example describes the selective hydrodeoxygenation of lauric acid to dodecane using various catalysts following the reaction procedure described in Example 3, with the exception that the conditions of 200° C. and 400 psig were held for 6 hours instead of 4 hours.

The catalysts in this example containing tungsten and Pt/alumina (alumina-supported platinum) were prepared following the procedure described in Example 4. A similar procedure was followed to prepare a 5% Mo on 2% Pt/alumina catalyst. These catalysts were then used in a lauric acid hydrodeoxygenation reaction under the conditions described in Example 3, except that the reaction conditions of 200° C. and 400 psig were held for 6 hours instead of 4 hours. The results of these reactions are listed in Table 4.

TABLE 4
Molar Selectivity and Molar Yield of Dodecane and Undecane from the
Conversion of Lauric Acid from a 6-Hour Reaction

| | Lauric Acid | Dodecane | | Undecane | |
|---|---|---|---|---|---|
| Catalyst | Conversion^{b} | Selectivity^{c} | Yield | Selectivity^{c} | Yield |
| 2% W on 2% | 88% | 82% | 71% | 5% | 4% |
| Pt/alumina^{a} | | | | | |
| 5% W on 2% | 100% | 63% | 62% | 3% | 3% |
| Pt/alumina^{a} | | | | | |
| 7.5% W on 2% | 99% | 40% | 40% | 2% | 2% |
| Pt/alumina^{a} | | | | | |
| 10% W on 2% | 100% | 82% | 82% | 3% | 3% |
| Pt/alumina^{a} | | | | | |
| 5% Mo on 2% | 98% | 50% | 50% | 3% | 3% |
| Pt/alumina | | | | | |

^{a}Results shown for tungsten-containing catalysts are an average of two reaction runs.
^{b}Percent moles of lauric acid that converted to products.
^{c}Percent moles of lauric acid that converted into dodecane or undecane.

The results listed in Table 4 indicate that the reaction conditions described in Example 3, but using a 6-hour period at 200° C. and 400 psig, allow hydrodeoxygenation of lauric acid to dodecane with low production of undecane byproduct. Specifically, catalysts with 2% platinum and 2%, 5%, 7.5%, or 10% tungsten were capable of yielding 40%-82% dodecane with 4% or less undecane yield.

Table 4 also indicates that a catalyst comprising 5% molybdenum and 2% platinum supported on alumina was effective at catalyzing lauric acid hydrodeoxygenation to dodecane with little production of undecane. This observation suggests that catalysts comprising platinum and molybdenum are useful for carrying out certain C₁₀₋₁₈ oxygenate hydrodeoxygenation processes as described herein.

Given the results described in Example 8-11, inclusion of a heteropolyacid or heteropolyacid salt would be applicable to the reaction procedure described in this Example.

### Example 7

### Selective Hydrodeoxygenation of Lauric Acid to Dodecane in a 20-cc Multi-Reactor System Using Various Solvent Conditions

This Example describes the selective hydrodeoxygenation of lauric acid to dodecane using catalysts containing tungsten and platinum following the reaction procedure described in Example 3, with the exception that different solvents and different amounts of lauric acid substrate were used.

The catalyst used in this example which contained 2% tungsten and 5% platinum was prepared following the procedures described in Examples 1 and 4. This catalyst was then used in a lauric acid hydrodeoxygenation reaction under the conditions described in Example 3, with the following exceptions. The reactions used 1 or 2 grams of lauric acid substrate instead of 0.20 grams lauric acid. Also, either hexadecane (1 g) alone, water (1 g), or no solvent was used, as opposed to using a ˜17:1 mixture of tetradecane and hexadecane as the solvent. The results of these reactions are listed in Table 5.

TABLE 5
Molar Selectivity and Molar Yield of Dodecane and Undecane from the
Conversion of Lauric Acid Using Different Reaction Solvents^{a}

| | Reaction | | | | | | |
|---|---|---|---|---|---|---|---|
| | Conditions | | Lauric | | | | |
| | Lauric | | Acid | Dodecane | | Undecane | |
| Catalyst | Acid | Solvent | Conversion^{b} | Selectivity^{c} | Yield | Selectivity^{c} | Yield |
| 2% W on | 2 g | none | 71% | 15% | 11% | 2% | 1% |
| 5% | | | | | | | |
| Pt/alumina | | | | | | | |
| 2% W on | 1 g | hexadecane | 81% | 28% | 23% | 3% | 2% |
| 5% | | | | | | | |
| Pt/alumina | | | | | | | |
| 2% W on | 1 g | water | 32% | 5% | 2% | 2% | 1% |
| 5% | | | | | | | |
| Pt/alumina | | | | | | | |

^{a}Results shown for each reaction are an average of two reaction runs.
^{b}Percent moles of lauric acid that converted to products.
^{c}Percent moles of lauric acid that converted into dodecane or undecane.

The results listed in Table 5 indicate that catalysts comprising tungsten and platinum perform well in hydrodeoxygenation reactions that utilize organic solvents such as hexadecane. However, reactions that do not comprise a solvent also work: 2% W on 5% Pt/alumina catalyst in the absence of solvent was able to catalyze a reaction that yielded 15% dodecane with only a 1% yield of undecane.

Based on this example, and Example 3 in which 2% W on 5% Pt/alumina catalyst was used in a solvent containing tetradecane and hexadecane, it is apparent that these and similar organic solvents can be used in certain of the C₁₀₋₁₈ oxygenate hydrodeoxygenation processes described herein.

Given the results described in Example 8-11, inclusion of a heteropolyacid or heteropolyacid salt would be applicable to the reaction procedure described in this Example.

### Example 8

### Selective Hydrodeoxygenation of Lauric Acid to Dodecane in the Presence of a Heteropolyacid in a 40-cc Reactor

This Example describes the selective hydrodeoxygenation of lauric acid to dodecane using a carbon-supported platinum catalyst prepared in a reaction containing the heteropolyacid, phosphotungstic acid (abbreviated herein as WPA). This process was carried out under conditions including a temperature of 200° C. and a pressure of 1000 psig.

6.0 g of lauric acid (Sigma Aldrich, St. Louis, Mo.; >99%, lot. no. MKBG4553V), 5.4 g of tetradecane (Alfa Aesar, Ward Hill, Mass.; 99+%, lot. no. E09Y007) (the solvent for this reaction), 0.6 g of hexadecane (Sigma Aldrich, 99.9%, lot. no. 26396JMV), 1.426 g (0.6 g dry weight) of carbon-supported 1% platinum catalyst from Johnson Matthey, Inc. (West Deptford, N.J.; JM #25 carbon support) and 0.174 g WPA (Sigma Aldrich Inc.) (WPA thus about 1.3% by weight of the reaction mix) were ground in a mortar and pestle and then added to a 40-cc Hastelloy® pressure reactor equipped with a magnetic stirrer. The reactor was purged with nitrogen gas three times by pressurizing the reactor to about 150 psig each time and then depressurizing it. The reactor was then purged with hydrogen gas three times by pressurizing the reactor to 150 psig and then depressurizing it.

After these purging cycles, the reactor was pressurized to about 250 psig of hydrogen and heated to 200° C. Once the reactor was equilibrated at 200° C., the reactor pressure was raised to the experimental set point of 1000 psig. After 6 hours at 200° C., the reactor was allowed to cool down to 50° C. and was held under those conditions overnight.

The next day, the reactor was opened and a sample was drawn for GC analysis before being resealed. The reactor was purged in a similar way to the previous day with three purges of nitrogen and three purges of hydrogen respectively finally achieving 250 psig of hydrogen in the reactor. The reactor was then reheated to 200° C. and the hydrogen pressure was raised to 1000 psig once the set point temperature was reached. After six hours, the reactor was allowed to cool down to room temperature and was held there overnight.

The third running day followed the previous day exactly including sample removal and reactor preparation. The sample was run for another 6 hours to make a total run time of 18 hours over the course of 3 days. A sample was collected and run on GC after the third running day and run through GC as shown below.

All the collected samples were diluted with tetrahydrofuran and filtered through a standard 0.2-micron disposable filter. The filtered samples were then analyzed by a GC/FID (gas chromatography/flame ionization detector) to identify the components thereof and to measure the concentrations of the reactants and products. The individual components were identified by matching the retention times of the components with those of certain calibration standards. The hexadecane that was included in the reaction was used as an internal standard to determine the concentrations of each of the individual components.

With respect to the final product of the reaction, the conversion of lauric acid was observed to be about 99%, while the molar yield of dodecane was about 73%. The molar yield was about 0% for dodecanol and lauryl laurate, and about 1% for undecane with the mass balance closing to approximately 76% of the mass accounted for.

These results demonstrate that a feedstock comprising the C₁₂ oxygenate, lauric acid, can be used to produce a linear alkane via a hydrodeoxygenation process that employs a carbon-supported platinum catalyst in the presence of a heteropolyacid under low temperature conditions. The hydrodeoxygenation process mostly produced a completely deoxygenated product (dodecane) with a small amount of certain by-products.

Specifically, the low production of undecane (C₁₁) demonstrates that only a very low level of carbon loss through lauric acid decarboxylation occurred during the process. The high level of dodecane produced with a relatively low amount of the side products dodecanol and lauryl laurate demonstrates that the process efficiently deoxygenated the carboxylic acid moiety of the lauric acid feedstock.

Thus, mixing a supported metal catalyst with a heteropolyacid provided a catalyst that efficiently catalyzed the conversion of an oxygenated carbon chain to a linear alkane with minimal chain length reduction in a hydrodeoxygenation process.

### Example 9

### Selective Hydrodeoxyqenation of Lauric Acid to Dodecane with Supported Platinum Catalysts Prepared in the Presence of a Heteropolyacid

This Example describes the selective hydrodeoxygenation of lauric acid to dodecane using various supported platinum catalysts prepared in the presence of the heteropolyacid, phosphotungstic acid (WPA). This process was carried out in a 20-cc multi-reactor system under conditions including a temperature of 200° C. and a pressure of 400 psig.

The hydrodeoxygenation reaction was performed in an Endeavor® reactor system containing eight stainless steel reaction vessels. Each vessel has a volume of about 25 mL and is equipped for mechanical stirring. A 20-mL glass vial is used as a liner in each reactor in this system.

0.10 g dry weight of a carbon- or alumina-supported platinum catalyst, and 0.05 g WPA were combined in a mortar and pestle, ground, and then added to a 20-mL glass vial used in one of the reaction vessels in the Endeavor® reaction system. This preparation represents a catalyst in which the weight of WPA used was 50% with respect to the weight of the supported platinum catalyst (listed in Table 6 as “50% of Cat loading WPA”). Other amounts of WPA were also used, such as 30% with respect to the weight of the supported platinum catalyst (i.e., 0.03 g WPA and 0.10 g supported platinum catalyst).

Table 6 lists other amounts of WPA in the catalyst such as “2% loading of W using WPA”. This was calculated as follows: calculate the tungsten (W) content of the WPA and use the amount of WPA that would provide a total mass of W that is 2% with respect to the weight of the supported platinum catalyst. For example, using 0.10 g of supported platinum catalyst, about 0.002 g W is required for a “2% loading of W using WPA”. Based on there being about 76.6 wt % W in one molecule of WPA, about 0.026 g of WPA is mixed with the catalyst to load 0.002 g W. This same type of calculation was used for preparing the “5% loading of W using WPA”, “10% loading of W using WPA”, “30% loading of W using WPA”, “50% loading of W using WPA”, and “100% loading of W using WPA” catalysts listed in Table 6.

After preparing the catalyst dry-mix, 0.20 g of lauric acid (Sigma Aldrich, >99%, lot. no. MKBG4553V), 1.71 g of tetradecane (Alfa Aesar, 99+%, lot. no. E09Y007) (the solvent for this reaction), and 0.10 g of hexadecane (Sigma Aldrich, 99.9%, lot. no. 26396JMV) were added to the vial containing the catalyst. The system was sealed and connected to a high pressure gas manifold. The reactor was purged with nitrogen gas four times by pressurizing the reactor to 400 psig each time and then depressurizing it. The reactor was then purged with hydrogen gas three times by pressurizing the reactor to 400 psig and then depressurizing it.

After the purging cycles, the reactor was pressurized to 100 psig of hydrogen and heated to 200° C. Once the reactor temperature reached 200° C., more hydrogen was added to the reactor to raise its pressure to the experimental set point of 400 psig. The reaction was carried out isothermally for four hours before switching off the heat and cooling the reactor down to below 50° C. For the entire length of the reaction, the pressure was maintained at the 400 psig set point by adding hydrogen whenever the pressure dropped below 399 psig.

After the reactor was cooled down, the glass vial used for the reaction was removed from the reactor and centrifuged at 2000 rpm for 5 minutes. The resulting sample was decanted off to separate the catalyst (solid sample) from the rest of the reaction mixture (liquid sample). The liquid sample was further diluted with tetrahydrofuran and filtered through a standard 0.45-micron disposable filter. The filtered liquid sample was then analyzed by a GC/FID to identify the components thereof and to measure the concentrations of the reactants and products. The individual components were identified by matching the retention times of the components with those of certain calibration standards. The hexadecane that was included in the reaction was used as an internal standard to determine the concentrations of each of the individual components. The results of these runs, each of which used a particular catalyst/WPA preparation, are provided in Table 6.

Thus, binding a metal catalyst to a heteropolyacid salt provided a catalyst that efficiently catalyzed the conversion of an oxygenated carbon chain to a linear alkane with minimal chain length reduction in a hydrodeoxygenation process. The mixing procedure should also be applicable using a heteropolyacid salt.

TABLE 6
Molar Selectivity and Yield of Dodecane and Undecane from the Conversion of
Lauric Acid Using Various Catalysts Prepared in the Presence of a
Heteropolyacid

| | Lauric Acid | Dodecane | | Undecane | |
|---|---|---|---|---|---|
| Catalyst | Conversion^{a} | Selectivity^{b} | Yield | Selectivity^{b} | Yield |
| 5% Pt/Carbon (JM #23) + | 99% | 74% | 73% | 7% | 6% |
| 50% of Cat loading WPA | | | | | |
| 5% Pt/Carbon (JM #26) + | 100% | 85% | 84% | 5% | 5% |
| 50% of Cat loading WPA | | | | | |
| 5% Pt/Carbon (JM #27) + | 46% | 2% | 1% | 3% | 2% |
| 50% of Cat loading WPA | | | | | |
| 1% Pt/Carbon (Englehard | 31% | 16% | 5% | 5% | 2% |
| SO 10273) + 50% of Cat | | | | | |
| loading WPA | | | | | |
| EVONIK 1% Pt + 0.1% Cu | 12% | 27% | 3% | 12% | 1% |
| on Carbon + 50% of Cat | | | | | |
| loading WPA | | | | | |
| EVONIK 5% Pt + 5% Bi on | 9% | 36% | 3% | 13% | 1% |
| Carbon + 50% of Cat | | | | | |
| loading WPA | | | | | |
| 1% Pt/Alumina (JM #33) + | 37% | 12% | 4% | 4% | 1% |
| 50% of Cat loading WPA | | | | | |
| 5% Pt/Alumina (JM #32) + | 79% | 49% | 39% | 5% | 4% |
| 70% of Cat loading WPA | | | | | |
| 5% Pt/AP R C3769 Alumina | 67% | 44% | 30% | 4% | 3% |
| (BASF) + 50% Cat loading | | | | | |
| of WPA | | | | | |
| EVONIK F 101 XR + 50% | 73% | 56% | 41% | 7% | 5% |
| Cat loading of WPA | | | | | |
| EVONIK F 101 KYF + 50% | 60% | 39% | 23% | 6% | 4% |
| Cat loading of WPA | | | | | |
| EVONIK F 1181 XR + 50% | 31% | 18% | 6% | 14% | 4% |
| Cat loading of WPA | | | | | |
| EVONIK F 1181 KYF + | 34% | 24% | 8% | 11% | 4% |
| 50% Cat loading of WPA | | | | | |
| 5% Pt/Alumina (JM #32) + | 99% | 83% | 83% | 6% | 6% |
| 50% of Cat loading WPA | | | | | |
| 5% Pt/Carbon (JM #23) + | 99% | 82% | 81% | 7% | 7% |
| 50% of Cat loading WPA | | | | | |
| 5% Pt/Carbon (JM #26) + | 99% | 86% | 85% | 5% | 5% |
| 50% of Cat loading WPA | | | | | |
| 5% Pt/Carbon (JM #23) + | 99% | 79% | 79% | 7% | 7% |
| 50% of Cat loading WPA | | | | | |
| 5% Pt/Carbon (JM #26) + | 99% | 98% | 98% | 6% | 6% |
| 50% of Cat loading WPA | | | | | |
| EVONIK F 101 XR + 50% | 89% | 62% | 55% | 6% | 6% |
| Cat loading of WPA | | | | | |
| EVONIK F 101 KYF + 50% | 76% | 60% | 45% | 6% | 5% |
| Cat loading of WPA | | | | | |
| 5% Pt/AP R C3769 Alumina | 97% | 98% | 95% | 5% | 5% |
| (BASF) + 50% Cat loading | | | | | |
| of WPA | | | | | |
| 5% Pt/Alumina JM #32 + | 45% | 24% | 11% | 6% | 3% |
| 2% loading of W using | | | | | |
| WPA | | | | | |
| 5% Pt/Alumina JM #32 + | 49% | 28% | 14% | 7% | 4% |
| 5% loading of W using | | | | | |
| WPA | | | | | |
| 5% Pt/Alumina JM #32 + | 59% | 44% | 26% | 6% | 4% |
| 10% loading of W using | | | | | |
| WPA | | | | | |
| 5% Pt/Alumina JM #32 + | 86% | 69% | 60% | 7% | 6% |
| 30% loading of W using | | | | | |
| WPA | | | | | |
| 5% Pt/Carbon JM #26 + 2% | 65% | 23% | 15% | 14% | 9% |
| loading of W using WPA | | | | | |
| 5% Pt/Carbon JM #26 + 5% | 84% | 30% | 25% | 12% | 10% |
| loading of W using WPA | | | | | |
| 5% Pt/Carbon JM #26 + | 79% | 37% | 29% | 10% | 8% |
| 10% loading of W using | | | | | |
| WPA | | | | | |
| 5% Pt/Carbon JM #26 + | 98% | 99% | 97% | 6% | 6% |
| 30% loading of W using | | | | | |
| WPA | | | | | |
| 1% Pt/Carbon (JM #25) | 76% | 75% | 57% | 3% | 3% |
| 30% loading of W using | | | | | |
| WPA | | | | | |
| 1% Pt/Carbon (JM #25) | 84% | 86% | 73% | 4% | 3% |
| 30% loading of W using | | | | | |
| WPA | | | | | |
| 1% Pt/Carbon (JM #25) | 76% | 88% | 67% | 3% | 3% |
| 50% loading of W using | | | | | |
| WPA | | | | | |
| 1% Pt/Carbon (JM #25) | 74% | 89% | 66% | 3% | 2% |
| 100% loading of W using | | | | | |
| WPA | | | | | |
| 5% Pt/Alumina JM #32 + | 63% | 9% | 5% | 3% | 2% |
| 300 μL Water + 50% Cat | | | | | |
| loading of WPA | | | | | |
| 5% Pt/Carbon JM #26 + | 99% | 69% | 68% | 18% | 18% |
| 300 μL Water + 50% Cat | | | | | |
| loading of WPA | | | | | |
| 5% Pt/Carbon JM #26 + | 99% | 72% | 72% | 18% | 18% |
| 300 μL Water + 50% Cat | | | | | |
| loading of WPA | | | | | |
| 5% Pt/Alumina JM #32 + | 44% | 6% | 3% | 3% | 1% |
| 300 μL Water + 50% Cat | | | | | |
| loading of WPA | | | | | |
| 5% Pt/Carbon JM #26 + | 98% | 81% | 79% | 16% | 16% |
| 600 μL Water + 50% Cat | | | | | |
| loading of WPA | | | | | |
| 5% Pt/Alumina JM #32 + | 14% | 3% | 0% | 6% | 1% |
| 600 μL Water + 50% Cat | | | | | |
| loading of WPA | | | | | |
| 5% Pt/Alumina JM #32 + | 6% | 6% | 0% | 12% | 1% |
| 600 μL Water + 50% Cat | | | | | |
| loading of WPA | | | | | |
| 5% Pt/Carbon JM #26 + | 97% | 67% | 65% | 27% | 26% |
| 600 μL Water + 50% Cat | | | | | |
| loading of WPA | | | | | |

^{a}Percent moles of lauric acid that converted to products.
^{b}Percent moles of lauric acid that converted into dodecane or undecane.

The results in Table 6 further demonstrate that a hydrodeoxygenation process employing a supported platinum catalyst that was prepared in the presence of a heteropolyacid can be used to produce a linear alkane from the C₁₂ oxygenate, lauric acid, under conditions of low temperature and pressure. The hydrodeoxygenation process mostly yielded the completely deoxygenated, full-length product, dodecane, with a very small amount of by-products. The low level of undecane produced indicates that there was little carbon loss during the process, while the low levels of dodecanol and lauryl laurate indicate that the carboxylic acid moiety of the lauric acid was efficiently deoxygenated.

Thus, dry-mixing a supported metal catalyst with a heteropolyacid provided a catalyst that could be used in a hydrodeoxygenation process to convert an oxygenated carbon chain to a linear alkane. The dry-mixing procedure should also be applicable using a heteropolyacid salt.

### Example 10

### Preparation of Heteropolyacid Cesium Salts

This Example describes the general procedure for preparing the partially Cs-exchanged heteropolyacids Cs_{2.5}H_{0.5}PW₁₂O₄₀ and Cs_{2.5}H_{0.5}SiW₁₂O₄₀. These heteropolyacid cesium salts were used in Example 11 to prepare various catalyst compositions comprising a heteropolyacid salt onto which platinum was loaded. The resulting catalyst compositions can be used in hydrodeoxygenation reaction procedures.

The cesium salts of the tungsten heteropolyacids were prepared using an aqueous solution of Cs₂CO₃ and an aqueous solution of either H₃PW₁₂O₄₀ or H₄SiW₁₂O₄₀. The heteropolyacid H₃PW₁₂O₄₀ or H₄SiW₁₂O₄₀ was prepared for use in aqueous solution by first dehydrating it at 60° C. under a vacuum for 2 hours. Cs₂CO₃ was dehydrated at 420° C. for 2 hours under a vacuum prior to its use for preparing an aqueous solution.

Partially Cs-exchanged heteropolyacids were prepared by titrating an aqueous solution of H₃PW₁₂O₄₀ (0.08 mol dm⁻³) or H₄SiW₁₂O₄₀ (0.08 mol dm⁻³) with an aqueous solution of Cs₂CO₃ (0.25 mol/L) at room temperature at a rate of 1 mL/minute. The resulting white colloidal suspension (precipitated heteropolyacid salt) was evaporated to a solid at 50° C. under a vacuum. The solids were then placed in a 120° C. vacuum oven for 2 hours to remove water. The dried solids were optionally calcined in air at 300° C. for 1 hour.

Thus, the partially Cs-exchanged heteropolyacids Cs_{2.5}H_{0.5}PW₁₂O₄₀ and Cs_{2.5}H_{0.5}SiW₁₂O₄₀ were prepared. Herein, Cs_{2.5}H_{0.5}PW₁₂O₄₀ is also referred to as phosphotungstic acid cesium salt (Cs—WPA). Example 11 below describes using Cs—WPA in preparing certain catalyst compositions for performing selective hydrodeoxygenation reactions.

### Example 11

### Selective Hydrodeoxygenation of Lauric Acid to Dodecane Using Catalysts Comprising a Platinum-Bound Phosphotungstic Acid Cesium Salt

This Example describes the selective hydrodeoxygenation of lauric acid to dodecane using catalysts that were prepared by loading platinum onto phosphotungstic acid cesium salt (Cs—WPA). This process was carried out in a 20-cc multi-reactor system under conditions including a temperature of 200° C. and a pressure of 400 psig.

An aqueous solution of ammonium platinum nitrate was used to load platinum, by wet-impregnation, onto Cs—WPA as prepared in Example 10. An appropriate amount of ammonium platinum nitrate solution was used to prepare catalysts in which 0.25%, 0.5%, 1.0%, or 2.0% by weight Pt was loaded on the Cs—WPA catalyst component. Wet-impregnation was performed on either calcined or non-calcined Cs—WPA (see Example 10). The wet-impregnated samples were dried at 100° C. under a slight vacuum, followed by calcination in a calcining furnace at 350° C. for 3 hours.

0.10 g of the calcined catalyst preparations were added to individual 20-mL glass vials used in the reaction vessels in the Endeavor® reaction system. Next, 0.20 g of lauric acid (Sigma Aldrich, >99%, lot. no. MKBG4553V), 1.71 g of tetradecane (Alfa Aesar, 99+%, lot. no. E09Y007) (the solvent for this reaction), and 0.10 g of hexadecane (Sigma Aldrich, 99.9%, lot. no. 26396JMV) were added to the vial containing the catalyst. The samples were run in the 20-cc Endeavor® reactor system and analyzed in the same way as described in Example 9. The results are summarized in Table 7.

TABLE 7
Molar Selectivity and Yield of Dodecane and Undecane from the
Conversion of Lauric Acid Using Platinum-Bound Phosphotungstic
Acid Cesium Salt

| | Lauric | | | | |
|---|---|---|---|---|---|
| | Acid | | | | |
| | Con- | Dodecane | | Undecane | |
| Catalyst | version^{a} | Selectivity^{b} | Yield | Selectivity^{b} | Yield |
| 0.25% Pt on | 91% | 89% | 81% | 3% | 3% |
| calcined Cs-WPA | | | | | |
| 0.5% Pt on | 95% | 92% | 88% | 4% | 4% |
| calcined Cs-WPA | | | | | |
| 1% Pt on calcined | 99% | 93% | 92% | 5% | 5% |
| Cs-WPA | | | | | |
| 2% Pt on calcined | 92% | 59% | 54% | 6% | 5% |
| Cs-WPA | | | | | |
| 0.25% Pt on non- | 99% | 68% | 68% | 4% | 4% |
| calcined Cs-WPA | | | | | |
| 0.5% Pt on non- | 99% | 96% | 95% | 4% | 4% |
| calcined Cs-WPA | | | | | |
| 1% Pt on non- | 99% | 93% | 92% | 4% | 4% |
| calcined Cs-WPA | | | | | |
| 2% Pt on non- | 99% | 93% | 91% | 6% | 6% |
| calcined Cs-WPA | | | | | |
| calcined Cs-WPA | 39% | 3% | 1% | 3% | 1% |
| non-calcined Cs- | 22% | 8% | 2% | 5% | 1% |
| WPA | | | | | |

^{a}Percent moles of lauric acid that converted to products.
^{b}Percent moles of lauric acid that converted into dodecane or undecane.

The results in Table 7 demonstrate that a hydrodeoxygenation process employing a platinum catalyst loaded on a heteropolyacid salt can be used to produce a linear alkane from the C₁₂ oxygenate, lauric acid, under conditions of low temperature and pressure. The hydrodeoxygenation process mostly yielded the completely deoxygenated, full-length product, dodecane, with a very small amount of by-products. The low level of undecane produced indicates that there was little carbon loss during the process, while the low levels of dodecanol and lauryl laurate indicate that the carboxylic acid moiety of the lauric acid was efficiently deoxygenated.

Thus, binding a metal catalyst to a heteropolyacid salt provided a catalyst that efficiently catalyzed the conversion of an oxygenated carbon chain to a linear alkane with minimal chain length reduction in a hydrodeoxygenation process.

What is claimed is:

1. A hydrodeoxygenation process for producing a linear alkane from a feedstock comprising a saturated or unsaturated C₁₀₋₁₈ oxygenate comprising a moiety selected from the group consisting of an ester group, carboxylic acid group, carbonyl group, and alcohol group, wherein the process comprises:
a) contacting said feedstock with (i) a catalyst comprising about 0.1% to about 10% by weight of a metal selected from Group IB, VIB, or VIII of the Periodic Table, and (ii) a heteropolyacid or heteropolyacid salt, at a temperature between about 150° C. to about 250° C. and a hydrogen gas pressure of at least about 300 psig, wherein the C₁₀₋₁₈ oxygenate is hydrodeoxygenated to a linear alkane, and wherein the linear alkane has the same carbon chain length as the C₁₀₋₁₈ oxygenate; and
b) optionally, recovering the linear alkane produced in step (a).

2. The hydrodeoxygenation process of claim 1, wherein said C₁₀₋₁₈ oxygenate is a fatty acid or a triglyceride.

3. The hydrodeoxygenation process of claim 1, wherein said feedstock comprises a plant oil or a fatty acid distillate thereof.

4. The hydrodeoxygenation process of claim 3, wherein said feedstock comprises
(i) a plant oil selected from the group consisting of soybean oil, palm oil and palm kernel oil; or
(ii) a palm fatty acid distillate.

5. The hydrodeoxygenation process of claim 1, wherein said catalyst comprises about 0.1% to about 6% by weight of platinum as the metal.

6. The hydrodeoxygenation process of claim 5, wherein said catalyst comprises about 0.25% to about 2% by weight of platinum as the metal.

7. The hydrodeoxygenation process of claim 1, wherein said heteropolyacid or heteropolyacid salt comprises tungsten.

8. The hydrodeoxygenation process of claim 7, wherein the heteropolyacid or heteropolyacid salt further comprises phosphorus.

9. The hydrodeoxygenation process of claim 1, wherein the heteropolyacid salt is a heteropolyacid cesium salt.

10. The hydrodeoxygenation process of claim 1, wherein said catalyst further comprises a solid support.

11. The hydrodeoxygenation process of claim 1, wherein the feedstock is contacted with the catalyst and the heteropolyacid salt, and wherein the catalyst is bound to the heteropolyacid salt.

12. The hydrodeoxygenation process of claim 1, wherein the catalyst and the heteropolyacid or heteropolyacid salt are dry-mixed together before they are contacted with the feedstock.

13. The hydrodeoxygenation process of claim 12, wherein the catalyst and the heteropolyacid are dry-mixed together before they are contacted with the feedstock.

14. The hydrodeoxygenation process of claim 1, wherein said temperature is about 200° C. and said pressure is about 400 psig.

15. The hydrodeoxygenation process of claim 1, wherein the molar yield is less than 10% for a reaction product having a carbon chain length that is one or more carbon atoms shorter than the carbon chain length of the C₁₀₋₁₈ oxygenate.
